(19) United States
(12) Patent Application Publication
(10) Pub. No.: US 2005/0129730 A1
(43) Pub. Date: Jun. 16, 2005

(54) Tissue composites and uses thereof

(75) Inventors: Roy H.L. Pang, Etna, NH (US); Robert A. Wiercinski, Lincoln, MA (US)

Correspondence Address: LAHIVE & COCKFIELD, LLP., 28 STATE STREET, BOSTON, MA, 02109, US

(73) Assignee: W.R. GRACE & COMPANY, Columbia, MD (US)

(21) Appl. No.: 10/958,550

(22) Filed: Oct. 4, 2004

Related U.S. Application Data
(63) Continuation of application No. PCT/US03/10439, filed on Apr. 4, 2003.
(60) Provisional application No. 60/370,043, filed on Apr. 4, 2002.

(51) Int. Cl. A61K 45/00; A61F 2/02

(52) U.S. Cl. 424/423; 424/93.7

(57) ABSTRACT

The invention is directed to improved tissue composites, e.g., biocompatible composites, that overcome or minimize the problems associated with existing tissue repair systems, which can be easily prepared and maintained in a sufficient quantity, and suitable shapes, to enable a convenient treatment of tissues requiring repair. Additionally, the invention is directed to methods of preparation of these tissue composites and methods of use thereof.

### RELATED APPLICATIONS

This application is a continuation of co-pending International Application No. PCT/US2003/010439, filed Apr. 4, 2003, which claims the benefit of Provisional Application Ser. No. 60/370,043, filed on Apr. 4, 2002, now abandoned. The contents of the above-referenced patent applications are expressly incorporated herein by reference in their entireties.

### BACKGROUND OF THE INVENTION

Injuries to soft tissues are extremely common in hospital clinics. In fact, soft tissue replacements amount to an estimated 35% of the world market for all medical devices (Materials Technology Foresight in Biomaterials, Institute of Materials, London (1995).

There have been many options proposed for the repair of soft tissues. These generally involve synthetic materials, biological materials or a combination of the two. Synthetic alternatives have demonstrated in vivo instability, and thus relatively poor long-term performance. Biological solutions traditionally involve autografts, allografts or xenografts, depending on the source of tissues. Each of these options has proved to be far from ideal with, for example, autografts leading to donor site morbidity, and allografts and xenografts to graft rejection.

In addition, despite advances in grafting techniques, skin grafting of denuded areas, granulating wounds and burns still present major healing problems. Split thickness autografts and epidermal autografts (cultured autogenic keratinocytes) have been used with variable success. However, both treatments have many disadvantages. For example, split-thickness autografts are generally unavailable in large body surface area (BSA) burns, cause further injury to the patient, and are of limited use in the treatment of patients with Dystrophic Epidermolysis bullosa (DEB). Furthermore, these autografts show limited tissue expansion, require repeated surgical procedures and protracted hospitalization, and give rise to undesirable cosmetic results. Epidermal autografts require time to be produced, have a low success (“take”) rate and often form spontaneous blisters. Additional limitations of epidermal autografts include fragility and difficulty in handling, contraction to 60-70% of their original size, and vulnerability during the first weeks following grafting. Significantly, such autografts have not proven useful in the treatment of deep burns where both the dermis and epidermis have been destroyed.

An alternative form of treatment is epidermal allografts (cultured allogenic keratinocytes), which has had some success in treatment of patients with second degree burns. The benefits of epidermal allografts include ready availability and quantities to provide treatment of patients in a single procedure, while avoiding autografting which increases the area of wounds and leaves painful infection-prone donor sites. In addition, the burn wounds treated with epidermal allografts demonstrate comparable healing rates to those treated with autografts, while also enabling the treatment of patients with DEB. Despite these advantages, epidermal allografts still experience many of the limitations of epidermal autografts. Moreover, full thickness skin injuries from burns that destroy both the epidermis and dermis, are still in need of treatment alternatives that replace both of these components.

The relative failure of many surgical, synthetic and graft solutions has led to a growing interest in the development of cell-seeded or tissue-engineered repair systems to address a number of clinical problems related to tissues, e.g., connective tissue or soft tissue. Such repair systems typically involve autologous or allogenic cells that are isolated from a tissue biopsy at a site remote to the injury. Typically, the isolated cells are expanded in cell culture and seeded in a suitable three-dimensional scaffold material, which when implanted into the injured site elicits a biological repair.

While previous studies have examined collagen sponges or foams for use as hemostatic agents, more recent attempts have examined collagen scaffolds for tissue repair in vivo, and as research tools in vitro for seeding various cell types in the study of cell function in three dimension (see e.g., U.S. Pat. No. 5,709,934). As collagen sponges have a low immunogenicity, and consist of a naturally occurring structural protein, cells can attach, interact with and degrade scaffolds of this type. The sponges are usually cross-linked to provide the degree of wet strength and measured resistance to dissolution needed for therapeutic efficiency. In general, however, cross-linking reduces or degrades the normal binding sites available to host cells and factors necessary of interactions with the scaffold following treatment. Furthermore, collagen sponges, gelatin sponges or polyvinyl alcohol sponges lack biological activity typically present in the extracellular scaffold environment of cells. In addition, existing biological dermal replacement composites generally require in vitro subculture before use.

Tissue-engineered systems for skin repair have been described in which fibroblasts are inoculated into a collagen scaffold, while keratinocytes are layered on a second non-porous collagen gel layer in contact with the collagen scaffold (see e.g., U.S. Pat. Nos. 6,039,760 and 5,282,859). Other constructs have been designed in which separate porous sponges are inoculated with different cell populations to produce a tissue construct. Constructs for cartilage replacement in which chondrogenic cells are cultured in a desired mold have also been described (see e.g., U.S. Pat. No. 5,786,217).

Alternative systems combine gel or hydrogel-cell compositions and support structures to form implantable tissue (see e.g., U.S. Pat. No. 6,306,169 and U.S. Pat. No. 6,027,744), in which the gel or hydrogel component substantially fills the support structure.

Interest in the area of tissue-engineered repair systems continues given the need for suitable skin equivalents, not only for repair of human or animal skin, for skin grafting, but also for determining the effects of pharmaceutical substances and cosmetics on skin.

### SUMMARY OF THE INVENTION

A need exists, therefore, for improved biocompatible composites that overcome or minimize the problems associated with existing tissue repair systems, and can be easily prepared and maintained in a sufficient quantity to enable convenient treatment of tissues requiring repair.

Accordingly, the invention is directed to improved tissue composites, e.g., biocompatible composites, that overcome or minimize the problems associated with existing tissue repair systems and can be easily prepared and maintained in sufficient quantities, and suitable shapes, to enable convenient treatment of tissues requiring repair. Additionally, the invention is directed to methods of preparation of these tissue composites and methods of use thereof.

One aspect of the invention pertains to a composite comprising a biocompatible porous scaffold in contact with a biocompatible gel seeded with cells. The biocompatible gel is in contact with at least one surface of the scaffold, and the scaffold and the gel form distinct compartments suitable for containing a biological material, for example a biological solution, e.g., a nutrient solution supportive of cell growth. A composite of the invention is schematically depicted in FIG. 1.

Another aspect of the invention pertains to a composite comprising a biocompatible porous scaffold in contact with a biocompatible gel seeded with cells. The biocompatible gel is in contact with at least one surface of the scaffold and the pores of the scaffold are substantially free of the gel.

Yet another aspect of the invention relates to a composite in which the porous scaffold is a biopolymer, e.g., collagen in the form of a particulate, dispersed within a biopolymer gel, e.g., collagen, seeded with cells. This embodiment of the invention is schematically depicted in FIG. 2. Optionally, the scaffold further includes a nutrient solution supportive of cell growth.

In yet another aspect, the invention pertains to a composite comprising a biopolymer scaffold in the form of a sheet, e.g., a planar sheet, in contact with a biopolymer gel seeded with cells. Optionally, the biopolymer scaffold further includes a nutrient solution supportive of cell growth.

In an embodiment particularly suitable for skin repair, the invention pertains to a composite comprising a collagen particulate scaffold dispersed within a collagen gel seeded with fibroblasts. In this embodiment, the particulate scaffold contains a nutrient solution supportive of cell growth. The composite can further include another cell population, for example, keratinocytes.

Yet another aspect of the invention suitable for skin repair pertains to a composite comprising a collagen particulate scaffold dispersed within a gel seeded with fibroblasts, in which the gel is selected from agarose and gelatin A, calcium alginate and gelatin A, and calcium alginate. In this embodiment, the collagen particulate scaffold contains a nutrient solution supportive of cell growth. The composite can additionally include other cell populations, for example, keratinocytes.

Other aspects of the invention feature composites including two or more cell populations. In one embodiment, the multi-cellular composite includes a collagen particulate scaffold dispersed within a first collagen gel seeded with a first cell population, e.g., fibroblasts. The collagen particulate scaffold contains a nutrient solution supportive of cell growth. The multi-cellular composite further includes a second collagen gel seeded with a second cell population, e.g., keratinocytes, in contact with at least one surface of the first collagen gel. A multi-cellular composite of the invention is schematically depicted in FIG. 3.

In another embodiment, the multi-cellular composite features two or more cell populations dispersed in distinct compartments. In a preferred embodiment, the multi-cellular composite includes a first collagen particulate scaffold dispersed within a first collagen gel seeded with fibroblasts. The first collagen particulate scaffold contains a nutrient solution supportive of cell growth. The multi-cellular composite further includes a second collagen particulate scaffold dispersed within a second collagen gel seeded with keratinocytes. The second collagen particulate scaffold contains a nutrient solution supportive of cell growth and is in contact with at least one surface of the first gel. This embodiment of the invention is depicted schematically in FIG. 4.

Other aspects of the invention feature multi-cellular composites in the form of a sheet. In a preferred embodiment, the multi-cellular composite includes a first collagen gel seeded with fibroblasts in contact with a first primary face of a collagen scaffold in the form of a sheet. The collagen scaffold contains a nutrient solution supportive of cell growth. The multi-cellular composite further includes a second collagen gel seeded with keratinocytes in contact with a second primary face of the sheet. This aspect of the invention is schematically depicted in FIG. 5.

Suitable biopolymers for use in the composites of the invention include collagen, a mixture of agarose and gelatin A, and complex coacervates such as calcium alginate and gelatin A, and calcium alginate. Preferred biopolymers for use in forming a porous scaffold include cross-linked biopolymers, e.g., collagen, having an average pore size that allows for cell growth and/or in-growth of cells, e.g., an average pore size of 1 to 100 microns, e.g., 2 to 50 microns, e.g., 2 to 20 microns or 20 to 50 microns.

Cell types for forming tissue composites of the invention include, for example, fibroblasts, keratinocytes, and stem cells. Cells for use in composites of the invention include primary cells, cultured cells and cryopreserved cells.

The present invention also pertains to use of the composites of the invention, including multi-cellular composites, in methods of treating a tissue or wound in a subject, in which the tissue or wound is contacted with a composite of the invention. In certain embodiments, subjects are treated following preparation and culture of the composite in vitro, to a desired cell density. In another embodiment, subjects are treated following preparation of the composite without culturing in vitro. In one embodiment, application of a composite to the subject occurs shortly after preparation, i.e., in vitro culturing is not required. Another aspect of the invention pertains to a method of preparation in which the composite can be prepared directly on the animal during treatment.

In addition, the present invention pertains to use of the composites of the invention, including multi-cellular composites, in methods of forming tissue or skin in a subject, in which the tissue or skin is contacted with a composite of the invention. In one embodiment, the tissue or skin is formed on the subject following preparation and culture of the composite in vitro to a desired cell density. In another embodiment, the tissue or skin is formed on the subject following preparation of the composite without culturing in vitro.

Other aspects of the invention feature methods of preparing composites of the invention in which at least one surface of a biocompatible porous scaffold is contacted with a biocompatible gel seeded with cells. In one embodiment, the biocompatible porous scaffold and the biocompatible gel are combined to form distinct compartments suitable for containing a biological material, thereby forming a composite. In another embodiment, a composite is prepared in which the pores of the biocompatible porous scaffold of the composite are substantially free of the biocompatible gel, thereby forming a composite.

Another aspect of the invention pertains to a method of preparing a composite comprising:
(a) wetting a biocompatible porous scaffold, e.g., a particulate biopolymer scaffold, with a biological material;
(b) preparing a dispersion of cells in a gellable biocompatible solution, e.g., a biopolymer solution; and
(c) contacting the wetted biocompatible porous scaffold with the gellable biocompatible solution under conditions suitable to gel the solution, thereby forming a composite.

An additional aspect of the invention pertains to a method of preparing a composite comprising a complex coacervate gel and a biopolymer scaffold. According to the method, a biopolymer scaffold is wetted with a nutrient solution that comprises a first component of the complex coacervate, e.g., calcium alginate. A biopolymer solution comprising a second component of the complex coacervate, e.g., gelatin A and cells is prepared and contacted with the wetted biopolymer scaffold, thereby forming a composite comprising a complex coacervate gel and a biopolymer scaffold.

Another aspect of the invention pertains to a multi-cellular composite. The multi-cellular composite comprises a collagen particulate scaffold dispersed within a first collagen gel seeded with a first population of cells, wherein the scaffold contains a nutrient solution supportive of cell growth; and a second population of cells, wherein the second population of cells is in contact with at least one surface of the first collagen gel.

Yet another aspect of the invention is directed to a multi-cellular composite comprising: a first collagen gel seeded with a first cell population in contact with a first primary face of a collagen sheet scaffold, wherein the scaffold contains a nutrient solution supportive of cell growth; and a second population of cells, wherein the second population of cells is in contact with a second primary face of the collagen sheet scaffold.

In an additional aspect, the invention relates to a method of preparing a multi-cellular composite. The method comprises contacting at least one surface of a biocompatible porous scaffold with a biocompatible gel seeded with a first population of cells under conditions suitable for gellation, thereby forming a single cell composite, and contacting the single cell composite with a second population of cells, wherein the scaffold, the gel, and the second population of cells form distinct compartments suitable for containing a biological material, thereby forming a multi-cellular composite.

In yet another aspect, the present invention is directed to a method of preparing a multi-cellular composite. The method comprises contacting at least one surface of a biocompatible porous scaffold with a biocompatible gel seeded with a first population of cells under conditions suitable for gellation, thereby forming a single cell composite, and contacting the single cell composite with a second population of cells upon gellation of the biocompatible gel, wherein the scaffold, the gel, and the second population of cells form distinct compartments suitable for containing a biological material, thereby forming a multi-cellular composite.

In another aspect, the invention is directed to a multi-cellular composite comprising at least one first multi-functional unit (MFU), and at least one second MFU. In this embodiment, the multi-cellular composite contains at least a first MFU that comprises a first biocompatible porous scaffold in contact with a first biocompatible gel seeded with a first population of cells wherein the gel is in contact with at least one surface of the scaffold.

Additionally, the present invention is directed a method of preparing a multi-cellular composite that comprises at least one first multi-functional unit (MFU), and at least one second MFU. The method comprises contacting at least one surface of a first biocompatible porous scaffold with a first biocompatible gel seeded with a first population of cells, thereby forming a first multi-functional unit (MFU), and contacting the first MFU with at least one second MFU, thereby forming a multi-cellular composite.

Yet another aspect of the invention pertains to a method of preparing a particulate porous collagen scaffold comprising:
(a) preparing an aqueous dispersion, e.g., about 0.05% to 10%, e.g., about 0.5% to 10%, of insoluble collagen at pH 1 to 5, e.g., 2 to 5;
(b) casting a droplet of the dispersion into a liquid medium at a temperature suitable to freeze the droplet;
(c) maintaining the frozen droplet under conditions suitable to lyophilize the droplet, thereby forming a collagen scaffold;
(d) exposing the collagen scaffold to conditions suitable to cross-link the collagen scaffold;
(e) wetting the collagen scaffold in a non-aqueous water soluble solvent, thereby forming a wetted cross-linked scaffold; and
(f) exposing the wetted cross-linked scaffold to a gradient of solvent mixtures comprising a non-aqueous solvent and an aqueous solution (e.g., water; a buffered and/or nutrient solution; or an aqueous solution suitable for maintaining cell viability and/or promoting cell growth) starting with a high concentration of the non-aqueous solvent and ending with the aqueous solution,
   thereby forming a particulate porous collagen scaffold. A composite prepared in accordance with this method is depicted in FIG. 6.

Yet another aspect of the invention is directed to a wetted particulate porous collagen scaffold prepared by the process of:
(a) preparing an aqueous dispersion, e.g., about 0.05% to 10%, e.g., about 0.5% to 10%, of insoluble collagen at pH 1 to 5, e.g., 2 to 5;
(b) casting a droplet of the dispersion into a liquid medium at a temperature suitable to freeze the droplet;
(c) maintaining the frozen droplet under conditions suitable to lyophilize the frozen droplet to form a collagen scaffold;
(d) exposing the lyophilized collagen scaffold to conditions suitable to cross-link the lyophilized collagen scaffold;
(e) wetting the cross-linked scaffold in a non-aqueous water soluble solvent, resulting in a wetted cross-linked scaffold; and
(f) exposing the wetted cross-linked scaffold to a gradient of solvent mixtures comprising the non-aqueous solvent and an aqueous solution (e.g., water; a buffered and/or nutrient solution; or an aqueous solution suitable for maintaining cell viability and/or promoting cell growth), starting with a high concentration of the non-aqueous solvent and ending with the aqueous solution.

In addition, another embodiment of the invention is directed to a wetted particulate suitable for containing a biological material comprising a porous cross-linked, e.g., dehydrothermally, collagen scaffold and an aqueous or non-aqueous solution, wherein the porosity of the particulate is substantially retained upon wetting.

Another aspect of the invention pertains to a method of identifying an agent, e.g., a pharmaceutical substance or cosmetic, that modulates cell growth, e.g., inhibit or increase cell growth in a composite of the invention. In certain aspects, the method involves contacting a composite of the invention with an agent to be tested and detecting a response by cells in the composite following contact with the agent.

### BRIEF DESCRIPTION OF THE DRAWINGS

FIG. 1 is a schematic representation of a biocompatible porous scaffold (1) in contact with a biocompatible gel (2) seeded with cells (3).

FIG. 2 is a schematic representation of a particulate biopolymer scaffold (4) dispersed in a biopolymer gel (5) seeded with cells (6).

FIG. 3 is a schematic representation of a multi-cellular composite comprising a collagen particulate scaffold (9) dispersed within a first collagen gel (7) seeded with fibroblasts (8) and a second collagen gel (10) seeded with keratinocytes (11) in contact with at least one surface of the first collagen gel.

FIG. 4 is a schematic representation of a multi-cellular composite comprising a first collagen particulate scaffold (14) dispersed within a first collagen gel (12) seeded with fibroblasts (13). The composite further includes a second collagen particulate scaffold (16) dispersed within a second collagen gel (15) seeded with keratinocytes (17), in contact with at least one surface of the first gel.

FIG. 5 is a schematic representation of a multi-cellular composite comprising a first collagen gel (21) seeded with fibroblasts (22) in contact with a first primary face of a collagen scaffold (20), in the form of a sheet, and a second collagen gel (18) seeded with keratinocytes (19) in contact with a second primary face of the sheet.

FIG. 6 is a confocal microscopy image depicting a collagen scaffold particulate pre-washed with ethanol prior to the addition of buffer, which illustrates that the scaffold maintains its structural integrity.

FIG. 7 is a confocal microscopy image depicting a collagen scaffold particulate directly washed with buffer, i.e., not pre-washed with ethanol prior to the addition of buffer, which illustrates a failure in the structural integrity of the scaffold, resulting in a reduction in diameter and significant reduction in pore size of the particulate.

FIG. 8 is a confocal microscopy image depicting a composite of the invention demonstrating proliferation of fibroblasts (23), in both the gel and the collagen scaffold (24) after incubation for 20 days.

FIGS. 9A-B are confocal microscopy images depicting composites of the invention with collagen particles, using a 20× objective. A solution of alginate and gelatin A was the gelling agent for the fibroblast layer. FIGS. 9A and 9B are images of the keratinocyte and fibroblast layer, respectively, after 4 days of incubation. One half of a million porcine keratinocytes were seeded in the keratinocyte layer while 3 million porcine fibroblasts were seeded in the fibroblasts layer on Day 0.

FIGS. 10A-D are confocal microscopy images depicting composites of the invention with collagen particles, using a 20× objective. FIGS. 10A and 101B are images of the keratinocyte surfaces after overnight and 5 days of incubation, respectively. FIG. 10C and 10D are images of the fibroblast surfaces after overnight and 5 days of incubation, respectively. One million porcine keratinocytes were seeded in the keratinocyte layer, while 3 million porcine fibroblasts were seeded in the fibroblast layer on Day 0.

FIGS. 11A-D are confocal microscopy images depicting composites of the invention with collagen particles, using a 20× objective. FIGS. 11A and 11B are images of the keratinocyte surfaces on Day 0 and 5 days of incubation, respectively. FIG. 11C and 11D are images of the fibroblast surfaces on Day 0 and after 5 days of incubation, respectively. One million porcine keratinocytes were seeded in the keratinocyte layer while 3 million porcine fibroblasts were seeded in the fibroblast layer on Day 0.

FIG. 12A-C are confocal microscopy images depicting longitudinal sections of composites of the invention with collagen particles, using a 5× objective. FIG. 11A, 11B and 11C are confocal microscopy images of the composites on Day 0, after overnight, and after 4 days of incubation, respectively. One million porcine keratinocytes were seeded in the keratinocyte layer while 3 million porcine fibroblasts were seeded in the fibroblast layer on Day 0.

FIG. 13 is a graph depicting the average size of wound sites determined during in vivo analysis of bi-layered composites on Day 3, 6, 8 and Day 14 after the implant of the composites.

Representations made in the figures are not intended to be limiting. Moreover, schematic representations were depicted as an even distribution of components of the composites of the invention for illustration only and are not meant to be limiting. In addition, relative sizes and shapes of components in the schematic depictions are not intended to be limiting on the scope of the invention.

### DETAILED DESCRIPTION OF THE INVENTION

I. Composites of the Invention

The invention is directed to improved tissue composites, e.g., biocompatible composites, that overcome or minimize the problems associated with existing tissue repair systems and can be easily prepared and maintained in a sufficient quantity, and suitable shapes, to enable convenient treatment of tissues requiring repair. Additionally, the invention is directed to methods of preparation of these tissue composites and methods of use thereof.

As used herein, the term “composite” includes a substantially solid material that is composed of two or more discrete materials each of which retains its identity, e.g., physical characteristics, while contributing desirable properties to the composite. For example, in certain embodiments of the invention, the composite is produced by two biopolymers each having independent physical characteristics, e.g., degree of cross-linking or porosity. Composites of the invention typically include a biocompatible scaffold and a biocompatible gel.

The term “scaffold” includes materials that provide a support structure, e.g., for cells or in-growth of cells, and are suitable for containing a biological material, e.g., a biological solution. In one embodiment of the invention, the scaffold is a biocompatible material, preferably a porous material, such as a porous biopolymer. In a preferred embodiment, the scaffold is a cross-linked biopolymer with an average pore size of about 1 to about 100 microns; preferably about 2 to about 50 microns, about 2 to about 20 microns or about 20 to about 50 microns. In certain embodiments of the invention, the scaffold has an average pore size that allows for cell growth and/or in-growth of cells. Preferably, the scaffold is a material that resists shrinkage and allows free flow of nutrients and waste throughout the material.

As used herein, the term “gel” includes materials that exist in a two-phase colloidal system consisting of a solid and a liquid in more solid form than liquid form, i.e., a semi-solid, of low porosity capable of retaining or immobilizing cells, while allowing the cells to proliferate. Accordingly, the gel is preferably formulated to allow diffusion of nutrients and waste products to, and away from cells, which promotes tissue growth following contact of a subject with a composite. In addition, the gel is preferably formulated to provide structural support to components of the composite, e.g., cells, during formation of the composite. The terms gel is intended to include materials that function as a “glue” to retain components of the composite in their desired location during formation of the composite as well as maintain the structural integrity of the composite following preparation and initial implantation in a subject. This aspect is particularly advantageous for composites in which the scaffold comprises particulates.

In addition, to maintain the structural integrity of the composite during the initial implant in a subject, in one embodiment the gel is a material that does not melt at 37° C. In one embodiment of the invention, the gel is a biocompatible material, preferably a biopolymer, such as collagen. In certain embodiments, the gel has a concentration of about 0.5 mg/mL to about 1.0 mg/mL of collagen, preferably, a concentration of about 0.6 mg/mL to about 0.9 mg/mL of collagen, or a concentration of about 0.6 mg/mL to about 0.72 mg/mL of collagen.

However, in one embodiment, it is desirable to reduce the structural integrity of the composite of the implant in a subject, e.g., partially liquefy the gel, at temperatures of about body temperature, to increase the rate of perfusion of nutrients to the cells thereby increasing the rate of cell proliferation and tissue remodeling. In particular embodiments the gel is a material that melts at 37° C. In specific embodiments, the gel remains as a gel at 30° C. in the composite.

For use in vivo, particularly in human subjects, it is preferred that the materials that compose the composite include materials that are biocompatible with the subject. The term “biocompatible” includes materials that are compatible with a subject and are not toxic or deleterious to the subject. In certain embodiments of the invention, the biocompatible material is biodegradable, such that it degrades or decomposes following contact with a subject, e.g., human. In a preferred embodiment, the biocompatible material is a biopolymer. Examples of commercially available biocompatible materials include collagen, e.g., type-I, -II, -III, and -IV, gelatin, alginate, agarose, e.g., type-VII, carrageenans, glycosaminoglycans, proteoglycans, polyethylene oxide, poly-L-lactic acid, poly-glycolic acid, polycaprolactone, polyhydroxybutarate, polyanhydrides, fibronectin, laminin, hyaluronic acid, chitin, chitosan, EHS mouse tumor solubilized extract, and copolymers of the above. However, the specific use of non-resorbable polymeric components, or of non-polymeric resorbable components such as soluble bioglasses is not precluded.

In certain embodiments of the invention, the composite is comprised of materials that are porous. The language “porous” includes materials having pores through which substances can pass. In certain embodiments of the invention, the scaffold component of the composite has an average pore size that allows for cell growth, for example, a porosity that allows nutrients and waste products to diffuse through the material. In another embodiment, both the scaffold and the gel components of the composite have an average pore size that allows for the in-growth of cells.

Preferred materials for use in composites of the invention are biopolymers. As used herein, the term “biopolymer” includes biocompatible materials composed of one or more polymeric materials that are typically formed in a biological system or synthetically prepared from biologically available monomers. A biopolymer of the invention can be in the form of a solid, semi-solid, or liquid, and can be isolated from a biological system or synthetically prepared. Additionally, biopolymeric solidification of a solution can occur, e.g., by aggregation, coagulation, coacervation, precipitation, ionic interactions, hydrophobic interactions, or cross-linking. In one embodiment of the invention, the biopolymer is a cross-linked biopolymer. Cross-linking may be induced chemically, thermally (e.g., dehydrothermal cross-linking), or by radiation, e.g., ultraviolet. Cross-linking agents for chemical cross-linking include but are not limited to glutaraldehyde, formaldehyde and like aldehydes; hexamethylene diisocyanate, tolylene diisocyanate, and like diisocyanates; ethyleneglycol diglycidylether, and like epoxides; and carbodiimide hydrochlorides. In a specific embodiment of the invention, the biopolymer is thermally cross-linked (e.g., dehydrothermal cross-linking). In a preferred embodiment of the invention, the biopolymer is a cross-linked collagen, for example, bovine Type I collagen.

Collagen for use in the composites of the invention is commercially available, for example, from Sigma Aldrich in a variety of forms. In addition, collagen may be extracted from animal tissue, e.g., bovine or porcine tissues, e.g., as described by Bell et al. in U.S. Pat. No. 5,709,934.

Alternative biopolymers for use in the composites of the invention include complex coacervates. The term “complex coacervate” includes an aggregate, e.g., of colloidal droplets, held together by electrostatic attractive forces. Additionally, the aggregate may be hydrated, i.e., comprising water. In certain embodiments of the invention, the complex coacervate comprises calcium alginate and gelatin A, or calcium alginate. In one embodiment of the invention, a complex coacervate gel is prepared by contacting a biocompatible porous scaffold comprising a first component of the complex coacervate, e.g., calcium alginate, with a biopolymer solution comprising a second component, e.g., gelatin A, of the complex coacervate. The combination of the two components upon the combination of the scaffold with the biopolymer solution acts to solidify the biopolymer solution through coacervation and aggregation.

Additionally, other biopolymers for use in the composite include agarose and mixtures of agarose and gelatin A. Preferably, the melting point for a gel comprising agarose and gelatin A is lower than for a gel comprising agarose alone. In a specific embodiment, the agarose mixture is a low temperature melting agarose.

The term “alginate” includes the salt or ester of an insoluble colloidal acid (C₆H₈O₆)ₙ, which in the form of its salts is a constituent of the cell walls of brown algae. In certain embodiments, the alginate exists as a calcium salt, and is thus termed a calcium alginate. Alginate is a polysaccharide, which can be derived from brown seaweeds, composed of D-mannuronic and L-glucuronic acid monosaccharide subunits. While the sodium salt of alginate forms viscous solutions, alginate can form hydrated gels in the presence of divalent cations such as calcium due to cross-linking through the negatively charged carboxyl groups residing on the L-glucuronic acid residues. The viscosity of the uncross-linked solutions and thereby the mechanical strength of cross-linked gels can be influenced by altering the average chain length of the alginate or by altering the proportion of D-mannuronic acid and L-glucuronic acid residues within the polysaccharide. These factors may also influence the rate of resorption of the alginate. Alginate is commercially available, for example, from Kelco International Ltd. Waterfield, Tadworth, Surrey, UK.

The term “gelatin” includes a variety of substances (such as agar) resembling gelatin, e.g., glutinous material obtained from animal tissues by boiling, e.g., colloidal protein used as a food, in the art of photography, and in the art of medicine. Gelatin A is prepared by briefly treating pigskins with dilute acid followed by extraction with water at 50-100° C. The resulting gelatin A has a high isoelectric point (pI), and thus is positively charged at physiological pH.

The term “agarose” includes a polysaccharide obtained from agar, e.g., known in the art as a common supporting medium in gel electrophoresis. Agarose is commercially available, for example, from Sigma, Poole, England.

A preferred embodiment of the invention is directed to a composite of a biocompatible porous scaffold and biocompatible gel, wherein the scaffold is substantially free of the gel. The language “substantially free of the gel” relates to an embodiment of the invention where the gel component of the composite surrounds, and does not substantially penetrate or fill the pores of the biocompatible porous scaffold. This can be accomplished by, for example, rapid solidification of a gelling agent to form a gel upon contact with a biocompatible porous scaffold. The language “substantially free of the gel” in relation to the porous scaffold, is not intended to include composites in which a gel-cell component penetrates a support structure, e.g., scaffold, of the composite, thereby substantially filling the support structure and taking the shape of the support structure.

Certain embodiments of the invention feature composites in which the porous biocompatible scaffold and the biocompatible gel form distinct compartments suitable for containing a biological material. The language “distinct compartments,” as used herein, relates to the ability of the components of the composite, i.e., the scaffold and the gel, to retain biological materials, for example, by immobilization or containment. In certain embodiments, cells are selected and positioned in the composite at desired locations to facilitate cell compartmentalization required for tissue repair and regeneration following implantation in a subject. As an exemplary embodiment, a composite for use in dermal wound repair is designed in which dermal and epidermal cells, e.g., fibroblasts and keratinocytes, are situated at desired locations in the composite to facilitate compartmentalization into dermis and epidermis following implantation in a subject, thereby forming new skin.

An advantage of the present invention is the ability to form distinct compartments in multi-cellular composites, e.g., composites containing two or more distinct cell populations, in a decreased amount of time as related to known tissue composites. For example, in certain embodiments a multi-cellular composite may be prepared in the time it takes a first gel containing a first cell population to harden to sufficient extent, such that a second cell population may be applied to the composite, e.g., a second cell population seeded into a second gel layer, or a second cell population without gel (wherein each of these second layers are intended to be considered as a distinct compartment from the first gel). Moreover, multi-cellular composites of the present invention may be prepared in less than about 6 hours, less than about 5 hours, less than about 4 hours, less than about 3 hours, less than about 2 hours. In certain embodiments, cells are selected and positioned on the composite at desired locations to facilitate cell compartmentalization required for tissue repair and regeneration following implantation in a subject. In particular, a multi-cellular composite can be prepared using a single gel layer that immobilizes a first population of cells, in combination with a second cell population layer that need not contain gel (i.e., the cells may be positioned on an exterior surface of the composite, i.e., directly in contact with the gel, and may adhere/adsorb to the composite/gel).

The language “multi-cellular composite” includes composites of two or more cell populations. In preferred embodiments of the invention, at least one of the two or more cell populations is seeded in gel in desired compartments in the composite such that the cell types are located to provide a specific tissue function in a subject. For example, in one embodiment of the invention, the first population of cells comprises fibroblasts and the second population of cells comprises keratinocytes.

The remaining cell populations may be seeded in gel or positioned on the exterior surface of the composite in a desired compartment of the composite such that the cell types are located to provide a specific tissue function in a subject. In certain embodiments of the invention, the gel is seeded with one cell population and the scaffold is seeded with a different cell population. In other embodiments, the gel is seeded with the same cell type that is contained in the scaffold. Alternatively, different cell types are in each of the gel and the scaffold. In one embodiment of the invention, the first population of cells comprises fibroblasts and the second population of cells comprises keratinocytes.

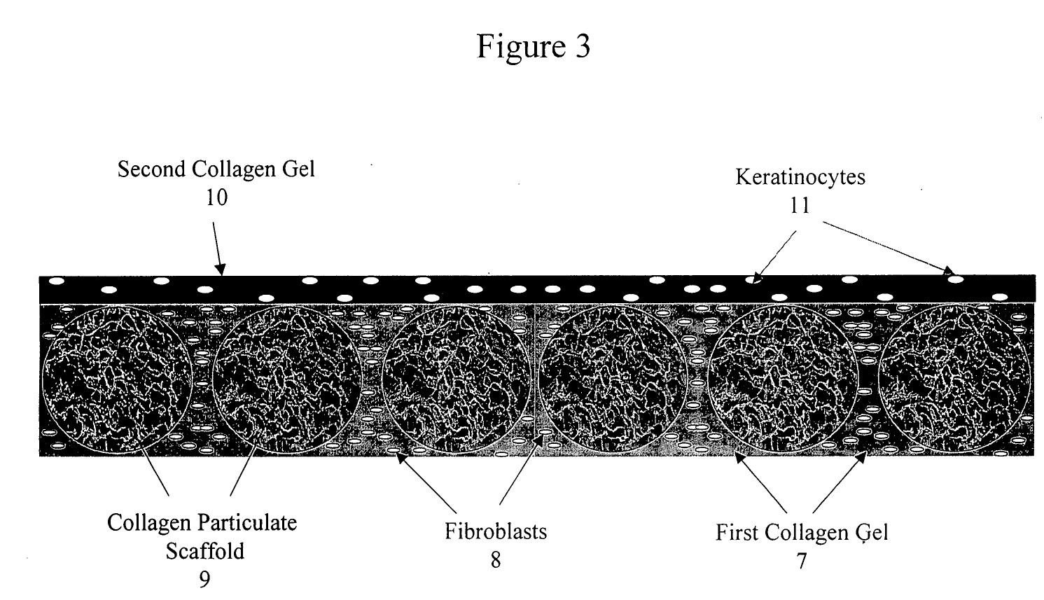

Another embodiment of the invention features multi-cellular composites having different cell types compartmentalized within the composite to facilitate formation of tissue, for example, at the site of a dermal wound. To prepare a tissue composite for treatment of a dermal wound, a gel containing a first population of dermal or epidermal cells, e.g., fibroblasts, is contacted with a porous scaffold, e.g., a particulate scaffold, thereby forming a tissue composite containing the dermal or epidermal cells. Subsequent to (e.g., immediately upon) gelling of the first gel, a second gel containing a second population of dermal or epidermal cells, e.g., keratinocytes, is positioned on at least one surface of the tissue composite containing the first population of dermal or epidermal cells, e.g., fibroblasts, thereby forming a dermal layer for use in tissue repair. A schematic representation of this embodiment of the invention is shown in FIG. 3. In addition, subsequent to (e.g., immediately upon) gelling of the first gel, the second population of cells may alternatively be positioned on at least one surface of the tissue composite containing the first population of dermal or epidermal cells without the need for the second gel to contain the cells.

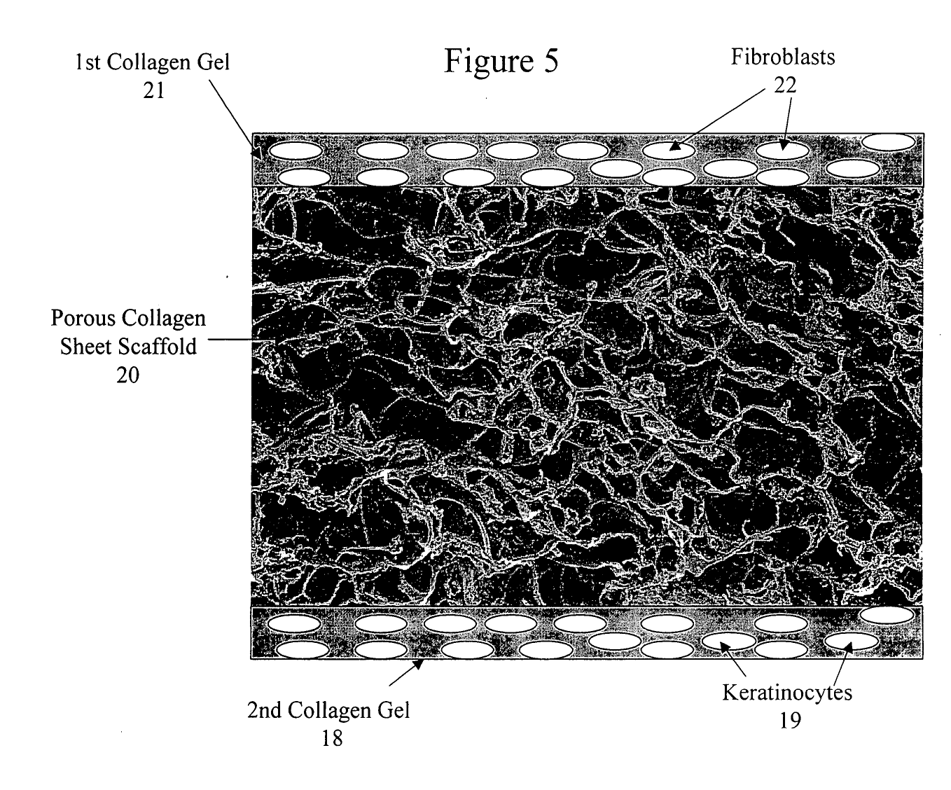

In an alternate embodiment, the porous scaffold is in the form of a sheet, and the first gel containing the first population of dermal or epidermal cells, e.g., fibroblasts is contacted with at least one surface of the scaffold. In this embodiment, immediately upon gelling of the first gel, the second gel containing the second population of dermal or epidermal cells, e.g., keratinocytes is contacted with an opposing surface of the scaffold, thereby forming a dermal layer for use in tissue repair. A schematic representation of this embodiment of the invention is shown in FIG. 5. In addition, immediately upon gelling, the second population of cells may alternatively be positioned on an opposing surface of the scaffold without the need for the second gel to contain the cells.

In another embodiment, the multi-cellular composite includes a first collagen particulate scaffold dispersed within a first collagen gel seeded with fibroblasts in contact with at least one surface of a second collagen gel seeded with keratinocytes. The collagen particulate scaffold of the multi-cellular composite additionally contains a nutrient solution supportive of cell growth. A schematic representation of this composite is shown in FIG. 3.

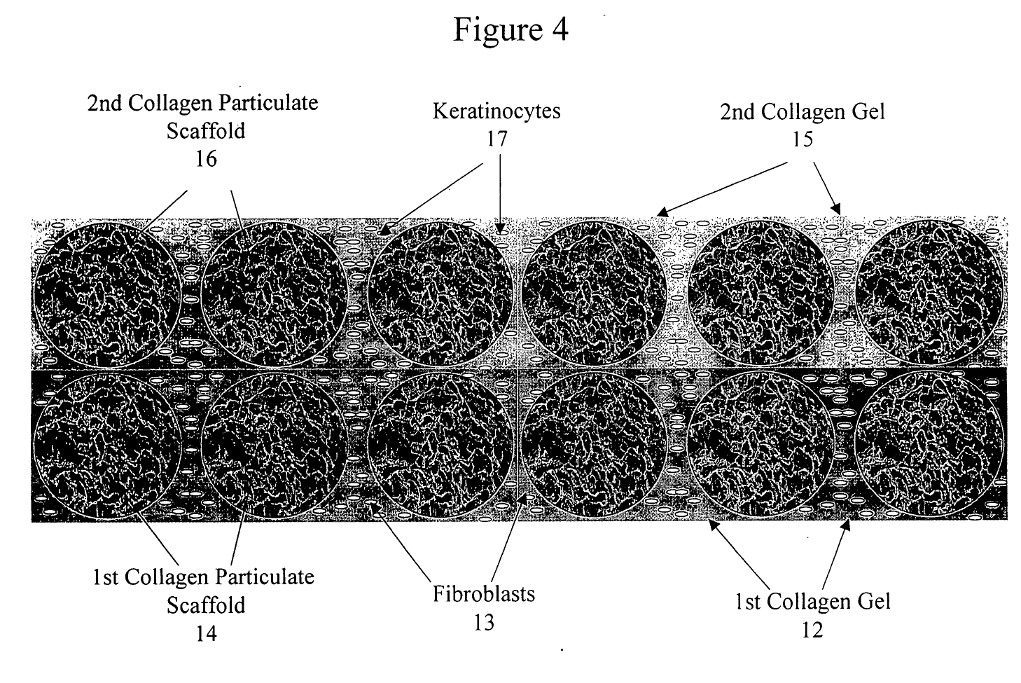

In yet another embodiment, the multi-cellular composite includes a first collagen particulate scaffold dispersed within a first collagen gel seeded with fibroblasts in contact with at least one surface of a second collagen particulate scaffold dispersed within a second collagen gel seeded with keratinocytes. Each collagen particulate scaffold of the multi-cellular composite contains a nutrient solution supportive of cell growth. A schematic representation of this composite is shown in FIG. 4.

Alternatively, the multi-cellular composite includes a first collagen gel seeded with fibroblasts in contact with a first primary face of a collagen scaffold, in the form of a sheet. The multi-cellular composite further includes a second collagen gel seeded with keratinocytes in contact with a second primary face of the collagen scaffold. The collagen scaffold optionally includes a nutrient solution supportive of cell growth. A schematic representation of this composite is shown in FIG. 5.

In another embodiment, the invention is directed to a multi-cellular composite comprising at least one first multi-functional unit (MFU), and at least one second MFU. In this embodiment, the multi-cellular composite contains at least one MFU that comprises a first biocompatible porous scaffold in contact with a first biocompatible gel seeded with a first population of cells wherein the gel is in contact with at least one surface of the scaffold.

Additionally, the present invention is directed a method of preparing a multi-cellular composite, which comprises at least one first multi-functional unit (MFU), and at least one second MFU. The method comprises contacting at least one surface of a first biocompatible porous scaffold with a first biocompatible gel seeded with a first population of cells, thereby forming a first multi-functional unit (MFU). This first MFU is then contacted with at least one second MFU, thereby forming a multi-cellular composite.

The language “multi-functional unit (MFU)” is intended to include distinct geographical and functional units (e.g., a unit with a distinct biological activity/function, e.g., a unit distinctly positioned for the growth of separate populations of cells) of a multi-cellular composite, wherein each functional unit may comprise a gel, a scaffold, a biological material, e.g., a cell population, or any combination thereof. For example, in certain embodiments of the invention, scaffold and gel combine to form one distinct multi-functional unit of a multi-cellular composite. In certain other embodiments, scaffold, gel, and cells are combined to form a single multi-functional unit. It should be understood that the inclusion of a biological material in a single MFU is not limited to a single biological material, e.g., a single MFU may contain more than one type of cell in a cell population.

In certain embodiments, the second MFU comprises a second population of cells in contact with at least one surface of the first MFU. In certain other embodiments the second MFU comprises a second gel seeded with a second population of cells in contact with at least one surface of the first MFU. In additional embodiments, the second MFU comprises a second biocompatible porous scaffold in contact with a second biocompatible gel seeded with a second population of cells wherein the second MFU is in contact with at least one surface of the first MFU.

The language “biological material” includes a material or agent that is biocompatible with a subject, e.g., a biological solution. Examples of biological materials include, but are not limited to water, buffered solutions, saline, nutrient solutions supportive of cell growth, cells, cell cultures, proteins, amino acids, cytokines, e.g., lymphokines, blood products, hormones, antibodies, e.g., monoclonal, toxins, toxoids, vaccines, e.g., viral, bacterial, endogenous and adventitious viruses, and pharmaceutical agents, e.g., pharmaceutical drugs. In one embodiment of the invention, the biological material is a biological solution.

The language “biological solution” includes biological materials, e.g., cells, in a liquid medium, e.g., aqueous solutions, e.g., water or buffered aqueous solutions. Biological solutions of the invention are prepared to allow easy delivery to, and storage within, the composite of the invention. In one embodiment, the biological solution is a nutrient solution supportive of cell growth.

The language “nutrient solution supportive of cell growth” includes solutions that contain nutrients, e.g., amino acids or growth factors supportive of cell growth. Optionally, the nutrient solution can contain cells.

For use in tissue repair, composites of the invention include one or more cell populations. Typically, the composite is seeded with cells of at least one cell type. The language “seeded with cells” includes a distribution of cells retained or immobilized within a material that contributes to the composite, e.g., the gel or scaffold. In certain embodiments, the distribution of cells is retained or immobilized in, for example, the gel, the scaffold, or both. The distribution of cells may be of a single type or of multiple types, e.g., as in the multi-cellular composites. In certain embodiments of the invention, the distribution of cells is a uniform distribution. In an embodiment where both the scaffold and the gel are seeded with cells, the cells may be selected for a specialized function in vivo (e.g., dermal and epidermal cells for skin repair) or be seeded with cells for independent function. Cells are selected and added to the material such that the composite can perform its intended function. Cells for use in the composites can be primary cells harvested from a donor, cultured cells, e.g., allowed to proliferate in vitro, or cryopreserved cells.

The language “cells contained in,” for example, in the expression, “the cells contained in the scaffold,” refers to a dispersion of cells in a biocompatible material, e.g., biopolymer, or adsorption of the cells and/or cell solution onto the surfaces of a biocompatible material. In contrast, the language “seeded with cells,” refers to retention, or immobilization, and placement of cells within a biological material.

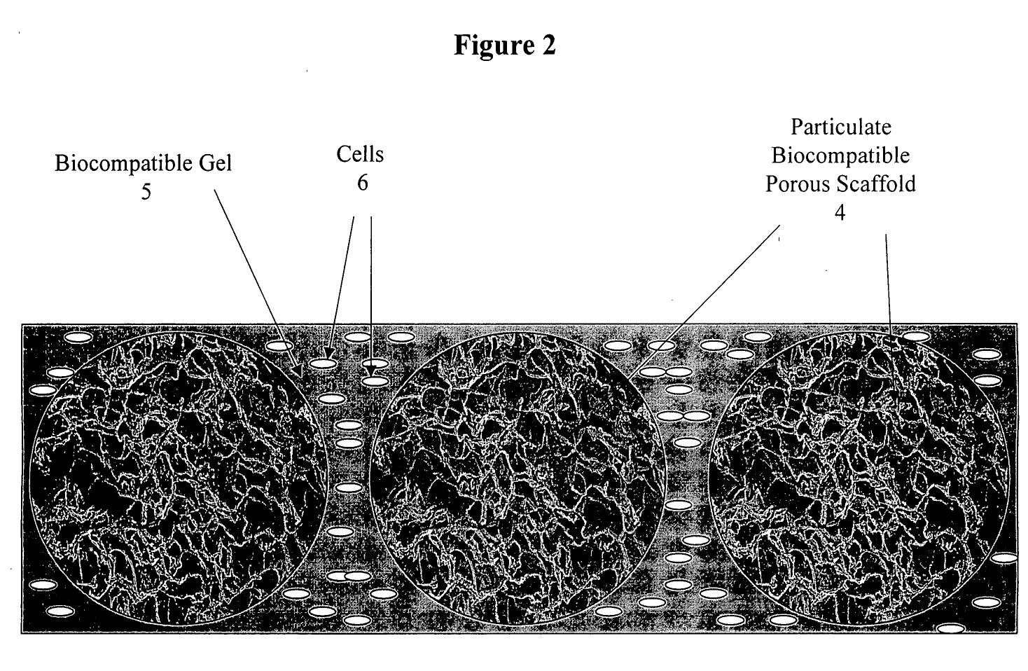

Preferred embodiments of the invention feature composites in which the components are particulate in nature. In one embodiment, the scaffold is in the form of a particulate. The term “particulate” as defined herein, includes materials, e.g., biopolymers, which are particle in nature, e.g., relatively minute, small, or discrete. The present invention is intended to include both spherical and non-spherical particulates. Moreover, particulates can be prepared as described in Example 1. Particulates can also be prepared according to art recognized techniques, e.g., U.S. Pat. No. 4,863,856, the contents of which are herein incorporated by reference. In certain embodiments, the particulates of the composite, e.g., the scaffold, are from about 0.1 mm to about 6.0 mm in diameter, about 0.1 mm to about 2.0 mm in diameter, or about 0.2 to about 1.3 mm in diameter; or preferably, about 0.5 to about 1.0 mm in diameter, about 1.0 to about 3.0 mm in diameter, or about 4.0 mm to about 5.0 mm in diameter. The effect of particulate size of the scaffold in minimizing shrinkage of the composite is described in Example 1 in section IV(b)(i). A composite in which the porous scaffold comprises particulates is shown schematically in FIG. 2.

Another embodiment of the invention is directed to a composite in the form of a sheet, e.g., planar sheet. In one aspect, the porous scaffold is in the form of a sheet. In another aspect, both the porous scaffold and the gel are in the form of a sheet. In yet another aspect, the gel is in the form of a sheet and the porous scaffold is in the form of a particulate. The term “sheet,” as used herein includes non-particulate materials, e.g., planar or three-dimensional, prepared from a mold. In certain embodiments, the sheet is a planar sheet. In a specific embodiment of the invention, the scaffold is a planar sheet and is in contact with at least one surface of the gel.

II. Methods of Preparation of the Composites of the Invention

Other aspects of the invention feature methods of preparing composites of the invention in which at least one surface of a biocompatible porous scaffold is contacted with a biocompatible gel seeded with cells. Following contact, the biocompatible porous scaffold and the biocompatible gel form distinct compartments suitable for containing a biological material, thereby forming a composite. In another embodiment, a composite is prepared in which the pores of the biocompatible porous scaffold of the composite are substantially free of the biocompatible gel, thereby forming a composite.

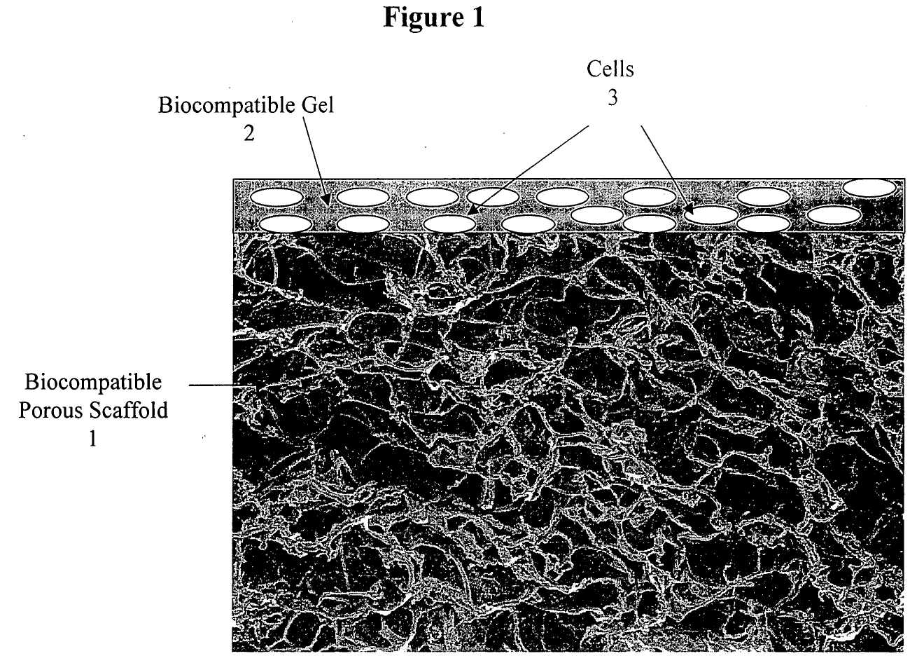

The language “contact” or “contacting” includes the union or junction of surfaces. The union may be made through a single point, in a region, i.e., surface, or in separate points or separate regions. The term “surface” as used herein includes the outer periphery, exterior, or upper boundary of a material. In certain embodiments, the term surface is used herein to describe a sheet structure, e.g., a scaffold in the form of a sheet, which is generally planar, e.g., a planar or curved, two-dimensional locus of points (as in the boundary of a three-dimensional region). In certain embodiments, contact of one surface is made with a primary face, e.g., a first primary face, of another surface. The language “primary face” includes surfaces of sheet structures that are comparatively larger than other surfaces of the sheet structure. Several examples of materials in contact are shown in FIGS. 1-5.

Accordingly, in one embodiment of the invention, a particulate scaffold is prepared as described in Example 1. For example, a particulate porous collagen scaffold is prepared from a 0.05% to 10% aqueous dispersion of insoluble collagen at pH 1 to 5. A droplet of this dispersion is then cast into a liquid medium at a temperature suitable to freeze the droplet and then the droplet is maintained under conditions suitable to lyophilize it to form a collagen scaffold. The lyophilized collagen scaffold is then exposed to conditions suitable to cross-link the lyophilized collagen scaffold. In a preferred embodiment, after cross-linking, the scaffold is wetted in a non-aqueous water-soluble solvent. The wetted cross-linked scaffold is subsequently exposed to a gradient of solvent mixtures comprising the non-aqueous solvent and buffer or nutrient solution, starting with a high concentration of the non-aqueous solvent and ending with buffer or nutrient medium, thereby forming a wetted particulate porous collagen scaffold. In certain embodiments, the non-aqueous water-soluble solvent is ethanol. In one embodiment, the particulate porous collagen scaffold may be initially wetted with absolute ethanol and then directly with the buffer or nutrient solution, i.e., creating a very steep two-step gradient. It should be understood that the novel wetted particulate collagen scaffolds, which retain their porosity upon subjection to wetting by utilization of this novel wetting protocol, are intended to be within the scope of this invention.

Accordingly, one embodiment of the invention is directed to a wetted particulate porous collagen scaffold prepared by the process of:
(a) preparing an aqueous dispersion, e.g., about 0.05% to 10%, e.g., about 0.5% to 10%, of insoluble collagen at pH 1 to 5, e.g., pH 2 to 5;
(b) casting a droplet of the dispersion into a liquid medium at a temperature suitable to freeze the droplet;
(c) maintaining the frozen droplet under conditions suitable to lyophilize the frozen droplet to form a collagen scaffold;
(d) exposing the lyophilized collagen scaffold to conditions suitable to cross-link the lyophilized collagen scaffold;
(e) wetting the cross-linked scaffold in a non-aqueous water soluble solvent, resulting in a wetted cross-linked scaffold; and
(f) exposing the wetted cross-linked scaffold to a gradient of solvent mixtures comprising the non-aqueous solvent and an aqueous solution (e.g., water; a buffered and/or nutrient solution; or an aqueous solution suitable for maintaining cell viability and/or promoting cell growth), starting with a high concentration of the non-aqueous solvent and ending with the aqueous solution.

In addition, another embodiment of the invention is directed to a wetted particulate suitable for containing a biological material comprising a porous cross-linked, e.g., dehydrothermally, collagen scaffold and an aqueous or non-aqueous solution, wherein the porosity of the particulate is substantially retained upon wetting. In certain embodiments of the wetted particulate, the average cross-sectional area, or volume or maximum diameter of wetted particulates, e.g., wetted with an aqueous medium, are within ±20% (preferably ±10%, more preferably ±5%) of the values for the dry precursors. In certain embodiments, the scaffold contains a biological material, e.g., biological solution, e.g., a nutrient solution supportive of cell growth (i.e., a nutrient solution that contains cells) or a pharmaceutical agent.

The lyophilized, cross-linked, scaffold can be directly wetted with buffer or nutrient solution, however this may cause shrinkage and collapse of the particulates of the scaffold, rendering the surface less porous. In addition, the surface of the particulates may collapse thereby rendering the surface less porous.

The term “casting” is well known in the art, and includes the process by which a material is formed into a shape to by pouring liquid into a mold and letting harden without pressure. In one embodiment of the invention, the hardening of the material is performed through temperature changes. In another embodiment of the invention hardening of the material is performed via complex coacervation. In certain embodiments of the invention, the casting of the scaffold is accomplished by exposure to low temperatures, e.g., liquid nitrogen.

The term “wetting,” is well known in the art, and includes the act of making a material wet. For example, in one embodiment of the invention involves the wetting of a biocompatible porous scaffold with a biological material, e.g., a biological solution.

A biocompatible gel can be prepared by addition of a gellable solution, prepared in accordance with the invention, to the scaffold or by addition of the scaffold to the gellable solution. In one embodiment of the invention, the gel is seeded with cells. In another embodiment, the gel rapidly solidifies to keep the cells at the application site, thereby eliminating problems of phagocytosis or cellular death and enhancing new cell growth at the application site. Alternatively, both the scaffold and the gel contain cells that are seeded in the material during preparation of the composite. In certain embodiments, the cells are added prior to gelling of the material.

The amount of gel used in the preparation of a composite is selected such that the resulting composite can perform its intended function. In certain embodiments, the volume fraction of the gel in relation to the scaffold particulates includes, but is not intended to be limited to a ratio of about 1:3 to about 1:1.6. Other ratios are also applicable to the present invention. A variety of cell types and numbers of cells can be selected based on the intended function and overall dimensions of the composites. In a preferred embodiment, about 1×10⁵ cells are combined with about 1.5 mL to about 2.0 mL of packed collagen particulates to form a composite of the invention.

The invention provides for various gelling agents, or gellable solutions, for the preparation of the tissue composites. These agents include soluble collagen gel, a cross-linked alginate/gelatin A complex in the presence of calcium ions or a low-temperature agarose/gelatin A mixture. The gelling agent is selected for the type of composite to provide optimal conditions for cell growth in the composite. The rate of gelation of each gelling agent can be controlled to facilitate the preparation of tissue composite of a desired shape. For example, in one embodiment of the invention, soluble collagen is combined with cells and maintained at 4° C. to retard the gelling process prior to mixing with collagen particulates that were maintained at either room temperature or 37° C. Consequently, the soluble collagen will gel on the surfaces of the collagen particulates with minimal or no gel penetrating into the collagen particulates. The temperature of the resulting mixture is then increased to 37° C. to facilitate the gelling process. In another embodiment of the invention, alginate and gelatin or agarose and gelatin are combined with cells and maintained at 37° C. during preparation. The mixture is then allowed to gel by incubation at 4° C.

An additional aspect of the invention pertains to a method of preparing a composite comprising a complex coacervate gel and a biopolymer scaffold. According to the method, a biopolymer scaffold is wetted with a nutrient solution that comprises a first component of the complex coacervate. A biopolymer solution comprising a second component of the complex coacervate is prepared and contacted with the wetted biopolymer scaffold, thereby forming a composite comprising a complex coacervate gel and a biopolymer scaffold.

Another aspect of the invention pertains to a method of preparing a composite comprising:
(a) wetting a biocompatible porous scaffold, e.g. a particulate biopolymer scaffold, with a biological material;
(b) preparing a dispersion of cells in a gellable biocompatible solution, e.g., a biopolymer solution; and
(c) contacting the wetted biocompatible porous scaffold with the gellable biocompatible solution under conditions suitable to gel the solution, thereby forming a composite.

The term “gelling,” is well known in the art, and includes the act of becoming solid or thickened by chemical or physical alteration, thereby changing into a gel.

Examples of cell types for use in forming tissue composites of the invention include but are not limited to epidermal and dermal cells (e.g., keratinocytes or fibroblasts), muscle cells (e.g., monocytes), cartilage cells (e.g., chondrocytes), bone forming cells (e.g., osteoblasts), epithelial cells (e.g., comeal cells, tracheal cells, or mucosal cells), endothelial cells, pleural cells, ear canal cells, tympanic membrane cells, peritoneal cells, gingiva cells, neural cells, hepatocytes, pancreatic cells, cardiac cells, and stem cells.

Cells for use in the composites of the invention can be isolated from a tissue biopsy or bone marrow sample from a subject, using methods known to those skilled in the art. If insufficient cell numbers are available at isolation, the cells can be allowed to proliferate in culture prior to seeding into a composite of the invention. During cell growth and proliferation, the cells can be cultured as a monolayer on a tissue culture treated substrate and maintained in tissue culture medium such as Dulbeccos Modified Eagle's Medium supplemented with, for example, between 1 and 20% fetal calf serum or autologous human serum. Alternatively, the cells can be cultured in serum free medium supplemented with mitogens on tissue culture plastic modified by the immobilization of specific attachment factors. In another approach, isolated cells can be seeded at a specified seeding density within alginate beads and cultured in tissue culture medium supplemented with serum or mitogenic growth factors. The cells can be isolated by dissolving the beads in a sodium citrate saline solution followed by collagenase digestion. The cells can be cultured within a suitable bioreactor.

In a particular embodiment for skin repair, cells are obtained from skin sample from a subject to be treated (autologous) or from donor tissue (allogenic). Skin samples are treated with trypsin to separate the epidermis from the dermis (Eisinger, M. Method in Skin Research, Editor D. Skerrow, (1985) pp 193). The epidermis is minced and treated with trypsin to release keratinocytes. The keratinocytes are then cultured until confluence using standard methods. In certain embodiments, the keratinocyte cells are cultured as single cell suspensions until confluence. Alternatively, in a preferred embodiment, the keratinocyte cells are seeded as single cell suspensions and cultured until confluence.

Primary cultures of fibroblast cells for use in accordance with the present invention may be prepared using standard methods such as, for example, the method disclosed in “A specific collagenase from Rabbit fibroblasts in monolayer culture,” Journal of Biochemistry (1974) 137, 373-385. Preferably, primary cultures of fibroblasts are prepared as follows. A dermal sample is cut up into 1 mm cubes and is suspended in a solution of collagenase buffered with Tris-HCl pH 7.4. A suitable collagenase is Clostridium histolyticum collagenase. The dermal sample is preferably suspended in solution at a concentration of 1 microgram/mL. The suspension is incubated and then centrifuged at 1,500 rev/sec to remove the cells from solution. The suspension is preferably incubated for 30 minutes. The cell pellet is washed with DMEM and the number of fibroblasts is determined with a haemocytometer. The viability of the fibroblast is determined by dye exclusion using Trypan Blue. The above culturing method also surprisingly yields other dermal epithelial cells that have a potential to develop into sweat glands or other skin cell types. An additional source of fibroblasts and keratinocytes includes neonatal foreskin, in which the cells can be isolated by standard protocols as described above.

Other embodiments of the invention involve the preparation of tissue composites of different shapes or forms using composites of the invention. The composite can be shaped to corresponded to the desired tissue to be formed, e.g., soft tissue, e.g., skin, bone, an organ, e.g., cartilaginous tissue, e.g., a meniscus for a knee, an ear, a nose, or other tissue. The shape of the composite may be equally affected by the shape of the individual components of the composite, i.e., the scaffold or the gel. Molding the composite to the desired shape can be achieved by selecting the shape of either the scaffold or the gel. In one embodiment, the shape of the composite is a product of a mold in which either the scaffold or the gel or both the scaffold and the gel are formed. For example, after mixing the desired cell types, the gelling agent and the collagen scaffold at a condition that will retard the gelling of the mixture, the mixture can be injected or cast into a mold of the desired structure under appropriate conditions to facilitate gelling of the mixture to the desired structure.

In another embodiment of the invention, a composite is prepared on the surface of a mesh to facilitate transfer to a subject. Preferred mesh comprises a polymer that is not bioabsorbable, preferably having a pore size ranging from 3 to 216 microns in diameter, as described in Example 1, IV(b)(i). In one embodiment, a nylon mesh is be used to reduce shrinkage of the composite, particularly with composites containing fibroblasts. It has been determined that shrinkage of the composite during in vitro culture is analogous to wound contraction in vivo, and therefore, the mesh and the desired size of the collagen particulates in the composite may be used advantageously in reducing wound contraction, if any, in vivo. Additionally, the mesh may be used to assist in handling of the composite prior to implantation in a subject or to assist in forming the composite into a desired shape.

III. Methods of Use of the Composites of the Invention

The present invention also pertains to use of the composites of the invention, including multi-cellular composites, in methods of forming a tissue or skin in a subject in which the subject is contacted with a composite of the invention. The invention also features methods of treating a tissue or wound in a subject in which the tissue or wound is contacted with a composite of the invention. In certain embodiments of the invention, the composite is prepared prior to application to the subject. In an alternative embodiment, the composite is prepared in situ. In one embodiment, subjects are treated following preparation of the composite without culturing of the composite in vitro. In certain embodiments, application of the composite to the subject occurs shortly after preparation, i.e., in vitro culturing is not required. In another embodiment, subjects are treated following preparation and culture of the composite in vitro to a desired cell density. Another aspect of the invention pertains to a method of preparation in which the composite can be prepared directly on the animal during treatment.

The terms “treating” and “treating a tissue or wound” are intended to include improving at least one condition of a tissue or wound, and tissue augmentation, i.e., plastic surgery, e.g., lip injections of composites.

The language “improving a condition of a tissue” includes growth of new tissue, protection of the tissue, e.g., from injury, e.g., infection, prevention of fluid loss, and tissue support to improve conditions for natural repair mechanisms of the subject. In one embodiment, contacting the tissue of a subject with a composite of the invention returns the tissue to a healthy state.

The term “tissue” includes cellular material capable of forming a collective entity. In one embodiment, a tissue is a collection or aggregation of morphologically and functionally similar cells. The term “wound” includes bodily injuries, including those which result in injury to a tissue, e.g., skin, e.g., a dermal wound. The language “subject” includes animals e.g., mammals, e.g., dogs, cats, horses, pigs, cows, sheep, goats, rodents, mice, rats, rabbits, squirrels, bears, and primates e.g., chimpanzees, gorillas, and humans, as well as transgenic non-human animals. Preferably, the subject is a human, e.g., a human requiring treatment of a tissue, e.g., wound repair.

A composite of the invention may be affixed to the patient through grafting techniques known in the art, for example, such as described by J. Hansbrough et al. (Journal of Med. Assoc., vol. 262, No. 15, Oct. 20, 1989 pp. 2125-2130. J. Hansbrough, S. Boyce, M. Cooper, T. Foreman Burn Wound Closure With cultured Autologous Keratinocytes and Fibroblasts Attached to a Collagen-Glycosaminoglycan Substrate). Additionally, the composite may be affixed to the subject through gelatinization, or lamination, as described by Morota et al. in U.S. Pat. No. 6,051,425.

An advantage of this invention includes the ability to implant a composite of the invention onto or into a subject directly after preparation without the prerequisite of in vitro culturing of the cells. In addition, during the proliferation of the cells in vivo, the kinetics of release, the types and the amounts of any factors produced by these cells released during cell proliferation in vivo will be available to the wound site, thereby expediting the wound healing process. Eliminating this culturing step reduces both the cost and time of production of tissue composites of the invention in comparison to known tissue repair systems.

In addition, eliminating the culturing step, eliminates the time required to wait before adding a second cell population to a composite in the preparation of a multi-cellular composite. Moreover, as described above, an advantage of the present invention is the ability to form distinct compartments in multi-cellular composites, e.g., composites containing two or more distinct cell populations, in a decreased amount of time as related to known tissue composites. For example, in certain embodiments a multi-cellular composite may be prepared in the time it takes a gel containing a first cell population to harden to sufficient extent, such that a second cell population may be applied to the composite, e.g., a second gel layer containing a second cell population or a second cell population without gel. Multi-cellular composites of the present invention may be prepared in less than about 6 hours, less than about 5 hours, less than about 4 hours, less than about 3 hours, less than about 2 hours.

It is contemplated that the composite can be conveniently prepared in less than 24 hours to be used on site or shipped off-site as required. In addition, since the product is shipped and used immediately after production, the requirement of maintaining an inventory of the final products may be eliminated, thereby reducing concern regarding the maintenance and shelf-life of the tissue composites. If desired, however, a composite of the invention can be prepared and cultured in vitro to a desired cell density prior to contacting the tissue or wound of the subject with the composite.

Another embodiment of the invention pertains to a method of identifying an agent that modulates a response by cells, e.g., cell growth or proliferation in a composite of the invention. In certain embodiments, the method involves contacting the composite with the agent and detecting a response by cells in the composite following contact with the agent. As used herein, the terms “modulate” or “modulation” include alteration of a response by cells, e.g., cell growth or proliferation in the composite, as compared to a response by cells in the absence of the agent. A response by cells includes, for example, cell growth or proliferation which can be modulated, e.g., increased or inhibited by an agent; a composite not contacted by an agent, e.g., alteration, e.g., inhibition or increase, of cell or tissue growth.

The term “agent” includes a product in the field of medicine, food, cosmetics, etc., which has been developed for direct application to a subject, e.g., a human, and therefore requires confirmation of the safety of the product. In the past, animal testing has been used as the main safety test, however, with drawbacks such as expense, long test periods, incomplete equivalence to humans, and public opinion for the prevention of cruelty to animals.

In this regard, skin equivalents also have been used as test skin for determining the effects of agents, such as pharmaceutical substances and cosmetics, on skin. In fact, a major difficulty in pharmacological, chemical and cosmetic testing is in determining the efficacy and safety of the products on skin. One advantage of the composites of the invention, is their use as an indicator of the effects produced by such substances through in vitro testing.

Exemplification of the Invention

The invention is further illustrated by the following examples, which should not be construed as further limiting.

### EXAMPLE 1

I. Preparation of Porous Collagen Scaffold

(a) Preparation of Collagen Suspension

(i) Alternative A

A suspension of insoluble bovine collagen (5 mg/mL) in 5.0% of glacial acetic acid was submitted to homogenization in a Silverson, lab scale, rotor/stator homogenizer for 1 minute at 4,000 rpm, followed by a 1 minute cooling interval at room temperature prior to each of 12 subsequent 1 minute bursts. The bovine collagen was subsequently incubated at 4° C. overnight.

(ii) Alternative B

A suspension of insoluble bovine collagen (5 mg/mL) in 5.0% of glacial acetic acid was submitted to homogenization in a Silverson, lab scale, rotor/stator homogenizer for 30 minutes at 6,000 rpm, while maintaining the temperature below 25° C. by chilling in ice bath. The bovine collagen was subsequently incubated at 4° C. overnight.

(b) Preparation of the Particulate Porous Collagen Scaffold

The insoluble bovine collagen suspension (200 mL) was allowed to pass through a 22-gauge needle into liquid nitrogen using a peristaltic pump. The collagen particulates were then incubated in the liquid nitrogen for an additional 5 minutes to ensure that the collagen particulates were completely frozen. After the particulates were lyophilized for 4-5 days, the collagen particulates were incubated in a vacuum oven at 120° C., for at least 3-4 days, to cross-link the collagen and sterilize the particulates.

(c) Preparation of the Sheet of Porous Collagen Scaffold

The insoluble bovine collagen suspension (5 mL) was pipetted onto a pyrex dish with a diameter of 5 cm. The collagen suspension was then frozen in liquid nitrogen or in a freezer at −20° C., lyophilized for 4-5 days, and subsequently incubated in a vacuum oven at 120° C. for at least 3-5 days, to crosslink the collagen and sterilize the collagen sheet.

II. Preparation of Non-Porous Gel for Immobilization of Cells onto Collagen Scaffold

(a) Soluble Collagen Gel

A nutrient solution was prepared as indicated in Table 1:

TABLE 1

| | |
|---|---|
| 10 × MEM | 16.2 mL |
| 10 × Glutamine/antibiotic Stock | 1.6 mL |
| Fetal calf serum | 18.0 mL |
| Sodium bicarbonate (71.2 mg/mL) | 5.0 mL |

A solution of acid soluble collagen was prepared or purchased as a 0.8-1.0 mg/mL solution in 0.05% acetic acid. The nutrition premix solution (1 mL) was mixed with the acid soluble collagen solution (3.5 mL) at 4° C. in a sterile 15 mL conical capped tube. After thorough mixing, the resultant solution is mixed with the desired cell type as described below for containment in composites.

(b) Alginate/gelatin A Gel

(i) Determination of the Melting Temperature of Cross-Linked Alginate and Gelatin A Complexes with Calcium Ions

Various concentrations of sodium alginate and gelatin A in 20 mM Tris.HCl, pH 7.0 were mixed together in Eppendorf tubes to a final volume of 0.5 mL, as indicated in Table 2. The temperature of the alginate and gelatin A solutions were maintained at 50° C. prior to mixing.

The mixtures were incubated at 4° C. for 2 hours to gel, and then overlaid with 0.2 mL of 0.5 M CaCl₂. The gel was subsequently incubated at 4° C. for 10 minutes, followed by the removal of the CaCl₂ solution. The melting temperature of the resultant complexes were determined by incubating the complexes at 30° C. for 10 minutes in a circulating water bath. The temperature was incrementally raised by 2° C., and incubated at the new temperature for 10 minute, until the temperature reached 58° C. The amount of gel liquefied at each temperature was observed after incubation at that temperature. The melting temperature of each complex, shown in Table 2, was the temperature at which 50% of the gel complex was liquefied.

TABLE 2

| | % Alginate | % Gelatin | Melting |
|---|---|---|---|
| Sample | (w/v) | A (w/v) | Temperature (° C.) |
| 1 | 0.5 | 5 | Not melted |
| 2 | 0.5 | 4 | 52 |
| 3 | 0.5 | 3 | 50 |
| 4 | 0.5 | 2 | 45 |
| 5 | 0.4 | 5 | Not melted |
| 6 | 0.4 | 4 | 49 |
| 7 | 0.4 | 3 | 49 |
| 8 | 0.4 | 2 | 40 |
| 9 | 0.3 | 5 | 45 |
| 10 | 0.3 | 4 | 41 |
| 11 | 0.3 | 3 | 39 |
| 12 | 0.3 | 2 | 37 |

As indicated in Table 2, the melting temperatures of the calcium alginate/gelatin A complexes can be controlled by varying the concentration of both calcium alginate and gelatin A. This facilitates the selection of the proper gel condition for immobilization of cells on the collagen particulates and provides the environment for the cells to proliferate.

(ii) Preparation of the Cross-Linked Alginate and Gelatin A Complex for Cell Immobilization

A cross-linked alginate and gelatin A complex was prepared by mixing alginate and gelatin A solution to a final concentration of 1.2% (w/v) and 6% (w/v), respectively, in 1×D-MEM, containing 10% fetal calf serum and 10 mM calcium chloride at pH 7.0 and 37° C.

The cross-linked alginate/gelatin A complex (0.4 mL) was mixed with D-MEM (0.2 mL) containing 10% fetal calf serum and (2×10⁵) normal human fibroblasts at 37° C. The resultant mixture was added to a well of a 24-well plate, which was then incubated at 4° C. for 4 hours to allow the alginate/gelatin A/cell mixture to gel. The cells were subsequently incubated at 37° C. in a CO₂ incubator, and culture medium was added, as necessary, to promote cell growth.

(c) Agarose/gelatin A Gel

(i) Determination of the Melting Temperature of Agarose and Gelatin A Complexes

Various concentrations of low temperature melting agarose and gelatin A in 20 mM phosphate buffer were mixed together in Eppendorf tubes to a final volume equal to 0.5 mL. The temperature of the agarose and gelatin A solutions were maintained at 50° C. prior to mixing.

After mixing, the agarose/gelatin A mixtures were allowed to gel at 4° C. for 2 hours. The melting temperature of the resultant complexes were determined by incubating the complexes at 30° C. for 10 minutes. The temperature was incrementally raised by 2° C., and incubated at the new temperature for 10 minute, until the temperature reached 54° C. The amount of gel liquefied at each temperature was observed after incubation at that temperature. The melting temperature of each complex, shown in Table 3, was the temperature at which 50% of the gel complex was liquefied.

TABLE 3

| | | | Melting |
|---|---|---|---|
| | % Agarose | % Gelatin | Temperature |
| Sample | (w/v) | A (w/v) | (° C.) |
| 1 | 2.0 | 0 | 52 |
| 2 | 2.0 | 0.5 | 50 |
| 3 | 2.0 | 5.0 | 51 |
| 4 | 1.0 | 0 | 45 |
| 5 | 1.0 | 0.5 | 43 |
| 6 | 1.0 | 5.0 | 31 |
| 7 | 0.5 | 0 | 41 |
| 8 | 0.5 | 0.5 | 38 |
| 9 | 0.5 | 5.0 | <30 |

As indicated in Table 3, the addition of gelatin A disrupted the agarose structure, thereby lowering its melting temperature. Based on this result, it was possible to determine an agarose/gelatin A mixture that will facilitate the immobilization of the cells on the collagen particulate and provides the proper environment for the cells to proliferate.

III. Effect of Ethanol on the Integrity of Porous Scaffolds

(a) Collagen Particulates

An average of about 10-20 collagen scaffold particulates were subjected to sequential washing with decreasing concentration of ethanol (EtOH) in phosphate buffer saline (PBS) in accordance with the washing conditions shown in Table 4a. In Groups I through IV, air was removed in vacuo during the first wash step in the solution indicated.

TABLE 4a

| Step | Group I | Group II | Group III | Group IV | Group V | Group VI |
|---|---|---|---|---|---|---|
| 1 | 100% EtOH | 100% EtOH | 100% EtOH | 70% EtOH | PBS | No wash |
| 2 | 70% EtOH | 50% EtOH | PBS | PBS | D-MEM | |
| 3 | 50% EtOH | PBS | D-MEM | D-MEM | | |
| 4 | 30% EtOH | D-MEM | | | | |
| 5 | PBS | | | | | |
| 6 | D-MEM | | | | | |

The collagen particulates were then transferred to D-MEM containing 10% fetal calf serum and 1× glutamine and penicillin/streptomycin. The diameters of each collagen particulates was subsequently measured. As shown in the Table 4b, the particulates from Group IV and V, in which ethanol was not used to wash the particulates prior to the addition of the PBS, collapsed after washing, and did not retain their spherical shape.

TABLE 4b

| Group | Average Diameters (in) |
|---|---|
| I | 2/32 |
| II | 2/32 |
| III | 4/32 |
| IV | Collapsed |
| V | Collapsed |
| VI | 3/32 |

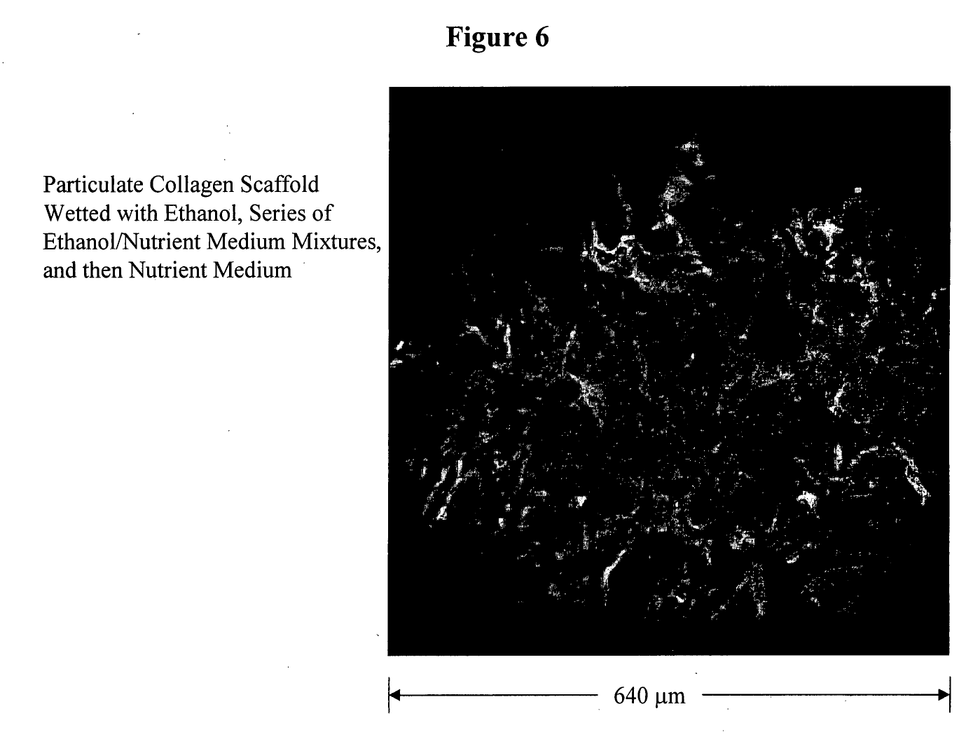

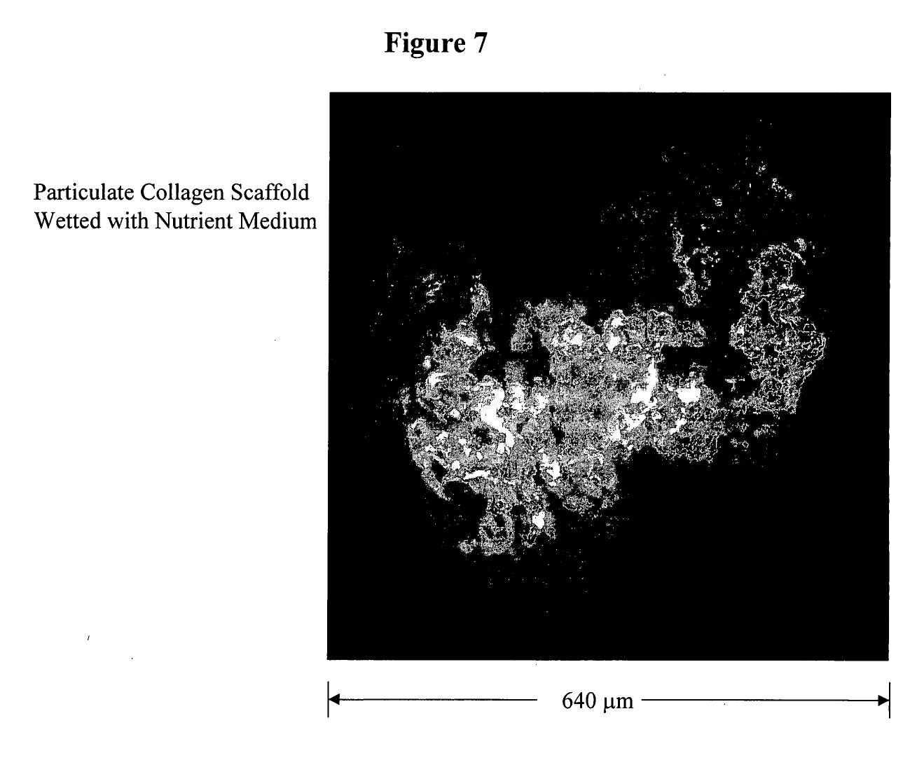

In addition, the particulates were stained and subjected to confocal analysis. As indicated in the confocal images, the surface of the Group I particulates was porous and maintained its integrity (FIG. 6), while the surface of the Group V particulates collapsed and was essentially non-porous (FIG. 7).

(b) Collagen Sheet

Collagen sheets were washed with ethanol as in Group I or Group V samples described above in Table 4a. After washing, the diameters of the collagen disks washed using the sequential steps of Group I, Table 4a, remained essentially the same as the dry samples, while those washed by the Group V protocol were reduced by about 40% of their original diameters.

IV. Preparation of Tissue Composite

(a) Preparation of Particulates for Tissue Composite

About 200 mL of dry collagen particulates were suspended in 200 mL of absolute ethanol, in a sterile 500-mL conical flask with a screw cap. The suspension was subjected to a vacuum to remove air in the particulates.

After the particulates sank to the bottom of the flask, the liquid was removed by first decanting, followed by using a pipette. About 200 mL of 70% ethanol in PBS was added to the flask, which was then shaken with a wrist shaker to mix the suspension until all the particulates sank to the bottom of the flask. The liquid was subsequently removed as previously described.

About 200 mL of 50% ethanol in PBS were then added, the suspension was shaken, and the liquid was removed after the particulates sank to the bottom of the flask. The process was repeated in accordance with Group I particulates of Table 4a, i.e., continuing with 30% ethanol in PBS, 100% PBS, and finally D-MEM containing 10% fetal calf serum supplemented with glutamine and penicillin/streptomycin. The particulates were stored in D-MEM at 4° C.

Additionally, the collagen sheet was also washed in a similar fashion and stored at 4° C. in D-MEM after washing.

(b) Tissue Composite with Soluble Collagen as the Gelling Agent

(i) Preparation of a Tissue Composite Using Collagen Particulates

Packed collagen particulates in culture medium (1.5 mL), prepared as described above, were pipetted into a single well of a 24-well plate. The single well may or may not contain a mesh with pore size ranging from 3 to 216 microns in diameter. Acid soluble collagen solution (0.35 mL), containing 1×D-MEM and 10% fetal calf serum at 4° C., was mixed with D-MEM (0.2 mL) containing 10% fetal calf serum and (1×10⁵) normal human fibroblasts at 4° C.

The excess culture medium of the collagen particulates in each well was removed and the collagen solution (0.45 mL) containing the cells was then mixed with the collagen particulates in the well of the plate, which was allowed to stand at room temperature.

The plate was then incubated at 37° C. in a CO₂ incubator to allow gel formation, thereby immobilizing the cells in a tissue composite. After gellation, 1 mL of culture medium was added to the well. The composite was allowed to remain at 37° C. in the CO₂ incubator, to demonstrate the ability for cell growth with medium changes, as needed, or used in an animal model for tissue repair.

Fresh culture medium was added to the composite every 4-5 days to promote cell growth. At time intervals indicated in Table 5, the composites were analyzed to determine size and cell growth, i.e., by confocal microscopy.

All of the composites shrank in size during incubation, mimicking in vivo wound contraction, however, the amount of shrinkage was greatly reduced in the composites prepared with a mesh at the bottom of the well, as indicated in Table 5. In general, in the absence of collagen particulates and mesh, the collagen gel containing the cells eventually formed a sphere of about 1-2 mm in diameter, demonstrating that the collagen particulates and mesh did, indeed, reduce shrinkage of the composite.

TABLE 5

| | |
|---|---|
| Time of | Size of Composite (mm) |

| Incubation (days) | No mesh | Plus Mesh |
|---|---|---|
| 0 | 15 | 15 |
| 22 | 9 | 14 |

The effect of the size of the collagen particulates on shrinkage was further investigated using collagen particulates with an average size of 1 and 2 mm, respectively. The composites were prepared as previously described in this section and the composites were incubated at 37° C. in a CO₂ incubator. The results are summarized in Table 6.

TABLE 6

| | Size of Composite (mm)* | |
|---|---|---|
| Diameter of | No Mesh | Plus Mesh |

| Particulate (mm) | Day⁺ 0 | Day14 | Day 0 | Day14 |
|---|---|---|---|---|
| 1 | 15 | 12 | 15 | 14 |
| 2 | 15 | 6 | 15 | 12 |

*Composite with fibroblasts and keratinocytes
⁺Time of incubation

The presence of mesh reduces shrinkage of the composites, as previously observed. In addition, the composite prepared with 1 mm diameter collagen particulates resisted shrinkage in the absence of the mesh. Therefore, both smaller collagen particulates and mesh may be used to reduces composite shrinkage during incubation in vitro and accordingly, will reduce wound contraction in vivo.

(ii) Preparation of Tissue Composite Using Collagen Sheet

A pre-wetted collagen sheet was placed in a single well of a 6-well plate with culture medium. The soluble collagen solution containing human fibroblasts was prepared as described in II(a). The soluble collagen solution (1 mL) containing 3×10⁵ fibroblasts was pipetted onto the collagen sheet in the well after removing the excess culture medium. The plate was incubated at 37° C. in a CO₂ incubator to facilitate the gelling of the collagen solution. Culture medium (3 mL) was then added and the composite was incubated at 37° C. in the CO₂ incubator, to demonstrate the ability of the composite to support cell growth (with culture medium replaced, as necessary) or for use in an animal model for tissue repair.

Similar to the ethanol-washed collagen particulates, the ethanol-washed collagen sheet composite contracted to about 60 to 70% of its original size during incubation, while the phosphate buffered saline-washed sheet composite remained in size.

(iii) Preparation of a Tissue Composite for Dermal Repair

A keratinocyte collagen solution was prepared by mixing keratinocyte culture medium (0.05 mL) containing (1×10⁵) human keratinocytes at 4° C., with a collagen solution (0.15 mL) prepared as described in II(a), at 4° C. Following gelation of the particulate collagen composite prepared as described in IV(b)(i), the resultant mixture was added to the top surface of the particulate collagen composite (which contained fibroblasts). The composite was then incubated at 37° C. to allow the collagen solution to gel. Subsequently, the composite was further incubated at 37° C. in a CO₂ incubator to facilitate the cell growth of both keratinocytes and fibroblasts, or used in an animal for tissue repair.

In a similar fashion to the composites described in IV(b)(i), the amount of reduction in size of the composites was decreased in the composite in which mesh was used during preparation (See Table 6).

Alternatively, the composites were prepared in inserts of a 24-well Falcon plate, containing a membrane on the bottom of the insert with a pore size of 3 microns. Keratinocyte culture medium (about 0.2 mL containing 1×10⁵ keratinocytes) was added to each insert, and then allowed to drain completely, leaving the keratinocytes on the membrane. Packed collagen particles (2 mL) in D-MEM supplemented with 10% fetal calf serum, 1× glutamine and 1× penicillin/streptomycin, were added slowly to the top of the keratinocytes to minimize disturbing the cells on the membrane, and the culture medium was then allowed to drain.

Collagen gel solution (0.2 mL), without cells, was then added onto the collagen particles and allowed to drain to the bottom of the insert to immobilize the keratinocytes at the bottom. After gellation at 37° C., collagen gel (0.45 mL), containing 1×10⁵ fibroblasts, was added to the top of the existing gelled composite. Keratinocyte culture medium (1.5 mL) was then added to the well and the resultant composite was further incubated at 37° C. to facilitate cell growth, or used in an animal for tissue repair.

(iv) Preparation of a Tissue Composite with Collagen Sheet for Dermal Repair

A collagen gel containing fibroblasts was prepared as described in IV.(b)(ii), and layered on one side of the collagen scaffold. The gelling solution containing the keratinocytes (1.25 mL) was then immobilized on the opposite side of the collagen sheet. After gellation, keratinocyte culture medium (2 mL) was added to each well and the composite was then incubated at 37° C. in a CO₂ incubator to demonstrate the ability of the composite to support cell growth (with culture medium replaced, as necessary) or for use in an animal model for tissue repair.

(b) Tissue Composite with Cross-Linked Alginate and Gelatin A Mixture

The tissue composites are prepared as described in III(a), except that the alginate/gelatin A mixture is used as a gelling agent.

(c) Tissue Composite with Agarose and Gelatin A Complex

For each composite, 7.5 mL of the washed particulates prepared as described in I(b) were pipetted into a sterile 6-well plate insert, with a diameter of 2.4 cm and a mesh (74 microns) at the bottom, which was in turn positioned in a sterile culture dish (10 cm in diameter). The medium in each insert was allowed to drain by gravity, and the particulates were then transferred to another sterile 6-well plate insert with a diameter of 2.4 cm and a 0.4 micron mesh at the bottom of the insert in a 10 cm diameter sterile culture dish, using a sterile spatula.

An alginate/gelatin A gelling solution was prepared by mixing the components listed in the following table. The components were maintained at 35° C. prior to mixing.

| Component | Concentration (%) | Volume (mL) |
|---|---|---|
| Alginate | 2 | 0.225 |
| Gelatin A | 15 | 0.360 |
| Deionized water | | 0.115 |
| Premix | | 0.200 |

0.55 mL of the alginate/gelatin A gel was mixed with 0.2 mL keratinocyte culture medium containing three million porcine fibroblasts and then pipetted into the particulates in the 6-welled plate insert. After mixing, the final concentration of alginate and gelatin A were 0.39% and 1.71%, respectively. The gel and particulates were mixed evenly using a pipette and the gel/particulate/cell mix was allowed to gel in the insert for half an hour at room temperature.

After gel formation, a collagen mixture solution was prepared by mixing 3.5 mL of soluble collagen solution and 1 mL of nutrition premix solution. The collagen mixture (0.35 mL) was added to 0.1 mL of keratinocyte culture medium containing one half of a million porcine keratinocytes and layered on top of the fibroblast/particulate/gel layer in the insert. The collagen solution was then allowed to gel at room temperature.

After gelling, the insert containing the gel, particulates and cells was transferred to a 6-well plate. Keratinocyte culture medium was added to the well as well as the insert to cover the composite. The composite was then incubated at 37° C. in a CO₂ incubator for 4 days. The alginate/gelatin A gel melted during incubation at 37° C.

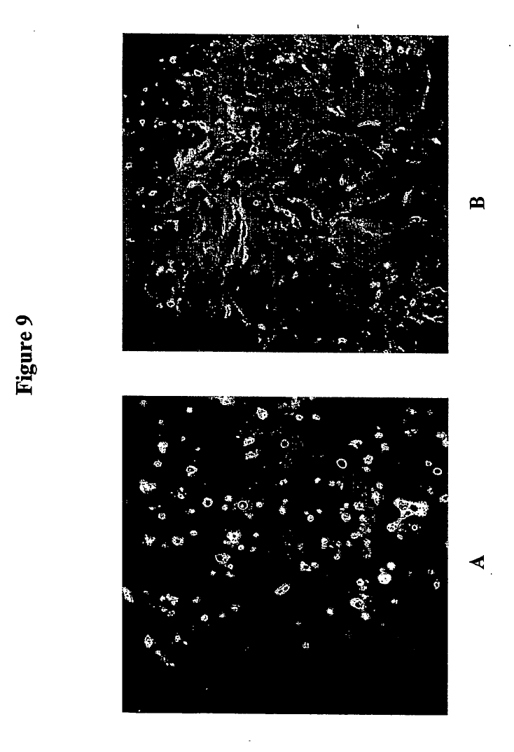

The composite in the insert was then washed with 1× phosphate buffered saline and then fixed with 10% formalin. The composite was then stained and analyzed by confocal microscopy. FIG. 9 shows the keratinocytes and fibroblasts in their respective surfaces.

V. Analysis of Cell Proliferation of Tissue Composites

At different time intervals of incubation at 37° C. in a CO₂ incubator, cell proliferation in the composites was analyzed by confocal microscopy. The diameter of the composite was determined to measure shrinkage and the composite was washed with phosphate buffered saline (3 mL) in a 15 mL capped conical tube. The phosphate buffered saline was then replaced with 2 mL of 10% buffered neutral formalin to fix the composite at room temperature for about 1-16 hours. After fixation, the formalin fixative was removed and the composite was washed three times with 3 mL of PBS (3-5 min at room temperature for each wash).

Alexa dye solution (0.5 mL), prepared by adding 20 μL of a stock solution (1 mg/mL) to 1 mL PBS, was added to the composite and allowed to stain for 30 minutes. After incubation, the stain solution was removed and the composite washed with PBS. Propidium iodide (0.5 mL of 1 mg/mL in PBS) was then added to the composite and allowed to stain for another 30 minutes. The stain was then removed and the composite was washed three times each with 3 mL of PBS (3-5 min at room temperature for each wash).

The resulting stained composite was stored in PBS at 4° C., if necessary, before analysis by confocal microscopy.

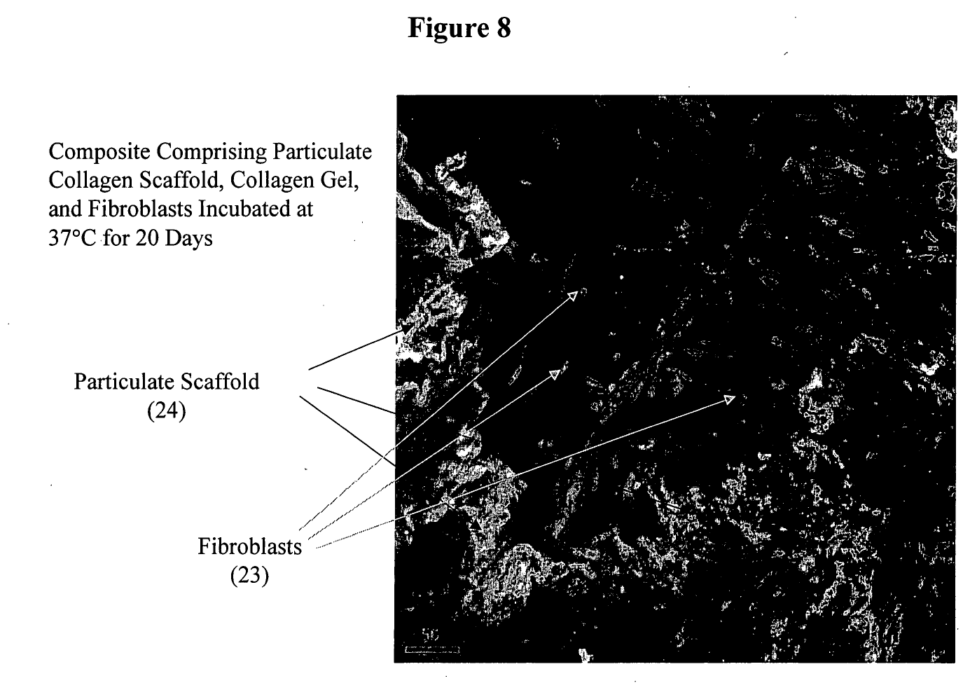

Moreover, FIG. 8 is a representative confocal image of a particulate composite to demonstrate cell proliferation in both the gel and the collagen matrix after incubation at 37° C. for 20 days, thereby showing that the composite supports cell growth. The cells on the surface of the scaffold appeared to be spindle-like, while the cells inside the scaffold appeared to be spherical. In addition, cells in this composite, which contain collagen particulates, are seeded throughout the composite in the inter-particular space. This is compared to a composite that contains a collagen sheet, wherein the cells are restricted to the surfaces of the matrix initially. As the cells are seeded throughout the composite, it is anticipated that this will facilitate more rapid wound repair in vivo providing the cells to the ability to remodel the whole composite simultaneously.

VI. In Vitro Preparation and Analysis of Bi-Layered Tissue Composites

Preparation of Bi-Layered Tissue Composites.

(i) Preparation of the Fibroblast Composite

About 200 mL of collagen particulates prepared as described in IV (a) and stored in D-MEM at 4° C. were transferred to a 500-mL filter apparatus with a 0.2 micron filter. The culture medium was removed by suction, and 200 mL of F12/D-MEM medium containing 15% of fetal calf serum; 2 mM glutamine; 1× penicillin/streptomycin; 0.39 mg/mL of L-arginine; 0.19 mg/mL sodium pyruvate; 2 μg/mL of putrescine; 8 μg/mL of insulin; and 8 μg/mL of hydrocortisone were added to the drained particulates. The particulates were then transferred to a sterile 500 mL bottle using a 25 mL pipette.

For each composite, 9 mL of the washed particulates, prepared as described in I(b), were pipetted into a sterile 6-well plate insert, with a diameter of 2.4 cm and a 74 microns mesh at the bottom, in a sterile culture dish (10 cm in diameter). The cultured medium in each insert was allowed to drain by gravity. The particulates were then washed with 10 mL of F12/DMEM, and again the medium was drained by gravity. After repeating the washing process one more time, the drained particulates were then transferred to another sterile 6-well plate insert with a diameter of 2.4 cm and a 0.4 micron mesh at the bottom of the insert in a 10 cm diameter sterile culture dish, using a sterile spatula.

The nutrition premix solution (1.0 mL) was mixed with 3.5 mL of collagen solution, containing 1.1 mg of collagen in acetic acid in a 15 mL sterile capped tube at 4° C. The gel solution (0.55 mL to 0.6 mL) was then mixed with 0.15 of mL of F12/DMEM medium containing 1 to 4 million fibroblasts obtained by trypsinization of a confluent culture of fibroblasts in a T75 tissue culture flask. The final volume of 0.7 to 0.75 mL of gel and cells was then pipetted into the insert containing the drained particulates.

Using a sterile 1 mL pipette, the particulates, gel and cells were mixed thoroughly by stirring. The composite inside the insert in a 10 cm diameter culture dish was then incubated at 37° C. for 5-10 minutes to allow the composite to gel.

(ii) Preparation of Bi-Layered Composite with a Keratinocyte Layer

After the gelling of the fibroblast composite described above, 25 mL of F12/DMEM were pipetted into the culture dish, but not into the insert. Then, 0.5 to 1.0 million of keratinocytes obtained by trypsinization of a confluent culture of keratinocytes grown in a Biocoat T75 flask coated with a layer of soluble collagen was suspended in 1.5 mL of culture medium and then loaded onto the top of the fibroblast composite in the insert. The composite was then incubated at 37° C. for 2 to 3 hours to allow the keratinocytes to attach to the top surface of the fibroblast composite. About 40 mL of culture medium were then pipetted into the culture dish to completely cover the composite in the insert. The composite was then incubated at 37° C.

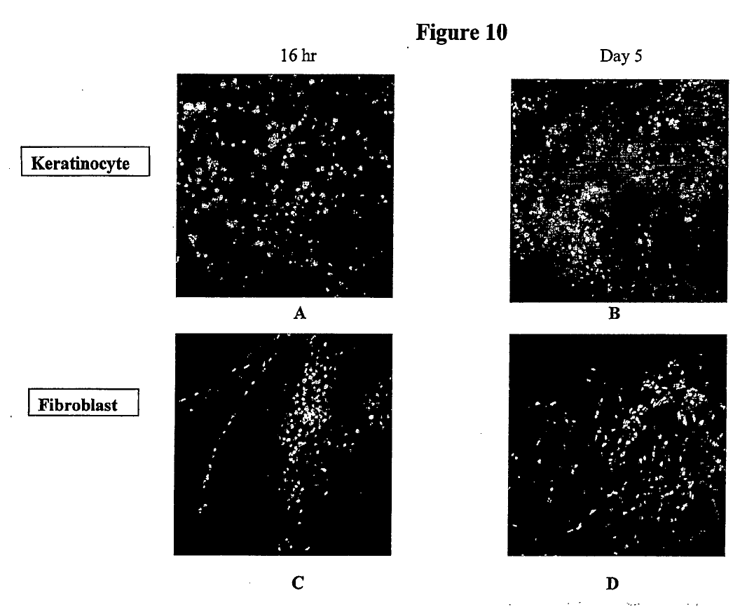

At the time indicated, the composite was removed, fixed in 10% formalin in 1× phosphate buffer saline, and analyzed by confocal microscopy after staining as described in Section V. FIG. 10 indicates confluent layers of keratinocytes on top of the bi-layered composites after 4-6 days of incubation. The confocal images were obtained using a 20× microscope objective. In addition, fibroblasts also proliferated as shown on the other sides of the composites.

(iii) Preparation of Bi-Layered Composite with Keratinocytes Embedded in a Collagen Gel

After gelling of the fibroblast composite described in VI (i), 0.35 mL of the premix/collagen gel was mixed with 0.1 mL of F12/DMEM medium containing 0.5 to 1.0 million keratinocytes obtained by trypsinization of a confluent culture of keratinocytes grown in a Biocoat T75 flask coated with a layer of soluble collagen. This mixture was then pipetted onto the fibroblast composite and the composite was again incubated at 37° C. for 5 to 10 min to allow the collagen to form a keratinocyte gel layer on top. The insert was then sealed in a Bio-Pak 250 mL container (CPL300) containing 60-80 mL of F12/DMEM medium, and the composites were then incubated at 37° C.

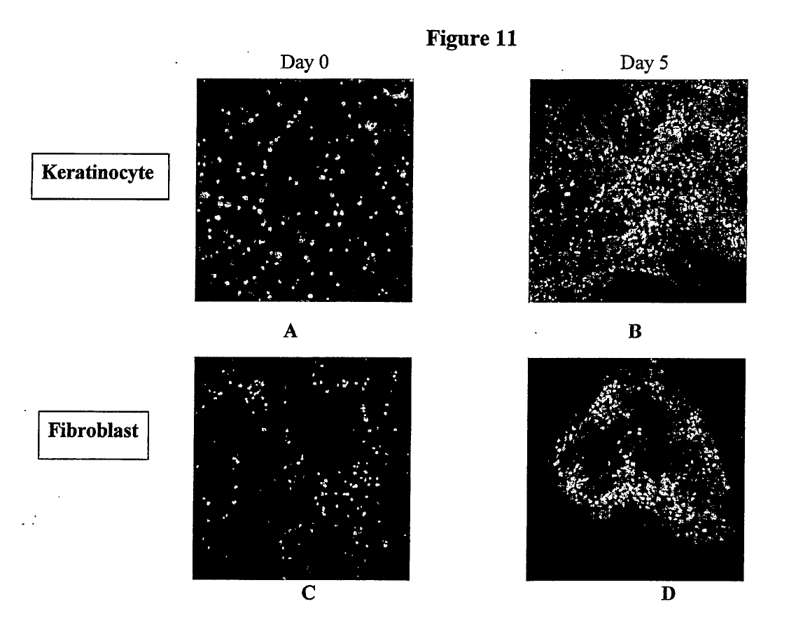

At the time indicated, the composite was removed, fixed in 10% formalin in 10% phosphate buffer saline and analyzed by confocal microscopy after staining as described in Section V. FIG. 11 depicts confluent layers of keratinocytes on top of the bi-layered composites after 4-6 days of incubation. The confocal images were obtained using a 20× microscope objective.

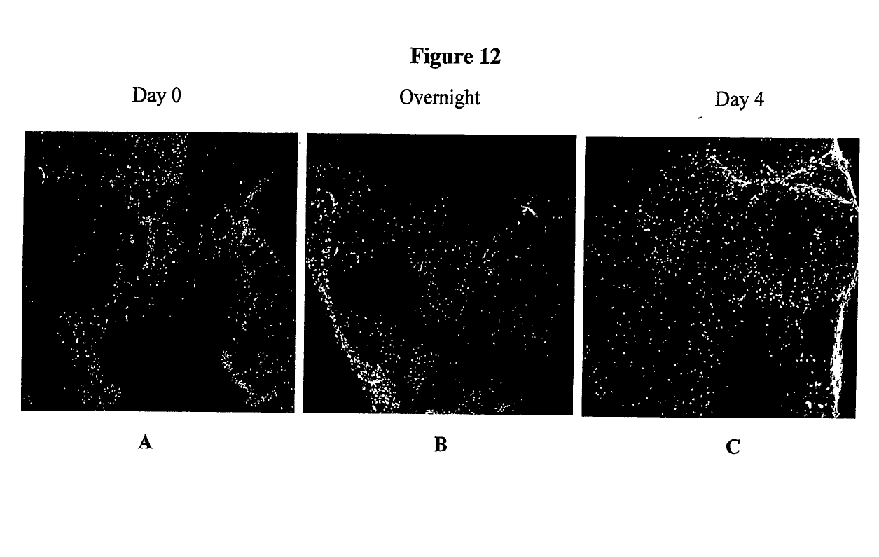

In addition, fibroblasts also proliferated as shown on the other sides of the composites. To ascertain fibrolast proliferation at the interior of the composites, longitudinal sections of the composite were imaged by confocal microscopy using a 5× objective. FIG. 12 indicates proliferation of fibroblasts after in vitro incubation.

VII. In Vivo Analysis of Bi-Layered Composites

(i) Preparation of Bi-Layered Composites

Porcine bi-layered composites were prepared as described in VI (iii). 15 mL of particulates were used for each composite. Three million allogenic porcine fibroblasts and 1 million of allogenic porcine keratinocytes were used in each composite, wherein the keratinocytes were embedded in a collagen gel. In addition, the bi-layered composites were prepared without fibroblasts and keratinocytes. The dimension of each circular composite was 2.4×2.4×0.6 cm. The composites in the inserts were incubated at 37° C. in 6-well plates with keratinocyte medium overnight.

(ii) In Vivo Analysis of Bi-Layered Composites

Eighteen full-thickness excisional square wounds (2.5×2.5×0.8 cm) were created on the dorsum of a Yorkshire pig weighing about 30 kg as described by Yao, F., et al. (Yao, F., et al. Age and growth factors in porcine full-thickness wound healing. Wound Repair and Regeneration. 2001; 372:371-377). Six (6) samples of each of the test groups: (a) bi-layered composites containing fibroblasts and keratinocytes, (b) bi-layered composites without cells and (c) saline control without composites, were used for the study.

Samples from each group were randomly implanted into the wound sites. The area of each wound site was determined by tracing the wound site onto a clear plastic sheet and scanning into a computer. The average size of the wound sites for each group was determined on Day 3, 6, 8 and Day 14 after the implant of the composites. The percent of contraction of the wound sites for each group was determined by the following equation:

$$\begin{matrix}Percent of wound \\ Contraction\end{matrix} = \frac{\begin{matrix}Size of original wound - \\ Size of wound at time indicated\end{matrix}}{Size of original wound before implant} \times 100$$

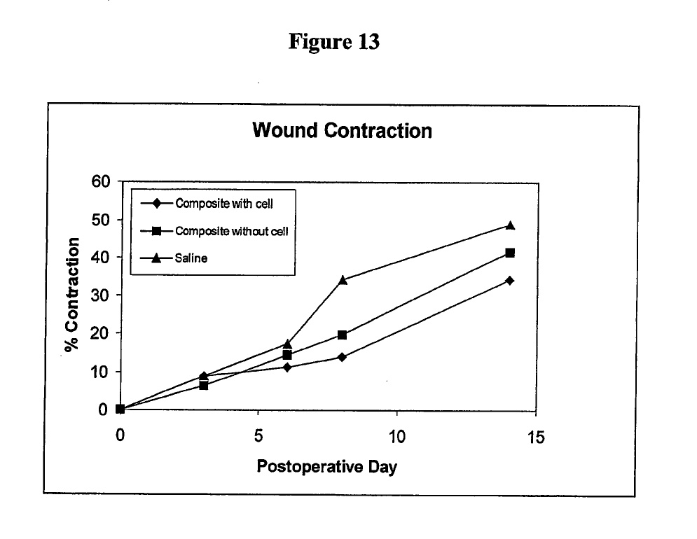

As indicated in FIG. 13, wound contraction was reduced using the composites of the present invention with or without cells when compared to the saline control at both Day 8 and 14 after implant. In addition, on both Day 8 and 14, the wound contraction is the least for the composites containing cells.

Equivalents

Those skilled in the art will recognize, or be able to ascertain using no more than routine experimentation, many equivalents to the specific embodiments and methods described herein. Such equivalents are intended to be encompassed by the scope of the following claims.

Incorporation by Reference

The entire contents of all patents, published patent applications and other references cited herein are hereby expressly incorporated herein in their entireties by reference.

1-159. (canceled)

160. A method of preparing a particulate porous collagen scaffold comprising:
(a) preparing an aqueous dispersion of insoluble collagen at pH 1 to 5;
(b) casting a droplet of the dispersion into a liquid medium at a temperature suitable to freeze the droplet;
(c) maintaining the frozen droplet under conditions suitable to lyophilize the frozen droplet to form a collagen scaffold;
(d) exposing the lyophilized collagen scaffold to conditions suitable to cross-link the lyophilized collagen scaffold;
(e) wetting the cross-linked scaffold in a non-aqueous water soluble solvent, resulting in a wetted cross-linked scaffold; and
(f) exposing the wetted cross-linked scaffold to a gradient of solvent mixtures comprising the non-aqueous solvent and an aqueous solution, starting with a high concentration of the non-aqueous solvent and ending with the aqueous solution,
thereby forming a particulate porous collagen scaffold.

161. The method of claim 160, wherein the aqueous dispersion of insoluble collagen is a 0.05% to 10% aqueous dispersion of insoluble collagen.

162. The method of claim 160, wherein the non-aqueous solvent is ethanol.

163. A wetted particulate porous collagen scaffold prepared by the process of:
(a) preparing an aqueous dispersion of insoluble collagen at pH 1 to 5;
(b) casting a droplet of the dispersion into a liquid medium at a temperature suitable to freeze the droplet;
(c) maintaining the frozen droplet under conditions suitable to lyophilize the frozen droplet to form a collagen scaffold;
(d) exposing the lyophilized collagen scaffold to conditions suitable to cross-link the lyophilized collagen scaffold;
(e) wetting the cross-linked scaffold in a non-aqueous water soluble solvent, resulting in a wetted cross-linked scaffold; and
(f) exposing the wetted cross-linked scaffold to a gradient of solvent mixtures comprising the non-aqueous solvent and an aqueous solution, starting with a high concentration of the non-aqueous solvent and ending with the aqueous solution.

164. The wetted particulate of claim 163, wherein the aqueous dispersion of insoluble collagen is a 0.05% to 10% aqueous dispersion of insoluble collagen.

165. A wetted particulate suitable for containing a biological material comprising a porous cross-linked collagen scaffold and an aqueous or non-aqueous solution, wherein the porosity of the particulate is substantially retained upon wetting.

166. The wetted particulate of claim 165, wherein the scaffold contains a biological material.

167. The wetted particulate of claim 166, wherein the biological material is a biological solution.

168. The wetted particulate of claim 167, wherein the biological solution is a nutrient solution supportive of cell growth.

169. The wetted particulate of claim 167, wherein the biological solution is a pharmaceutical agent.

170. The wetted particulate of claim 168, wherein the nutrient solution contains cells.

171. The wetted particulate of claim 165, wherein the porous scaffold contains pores with an average pore size that allows for cell growth.

172. The wetted particulate of claim 165, wherein the porous scaffold contains pores with and an average pore size that allows for the in-growth of cells.

173. The wetted particulate of claim 165, wherein the cross-linked collagen porous scaffold is thermally cross-linked

174. The wetted particulate of claim 165, wherein the porous scaffold has an average pore size of 1 to 100 microns.

175. The wetted particulate of claim 165, wherein the porous scaffold has an average pore size of 2 to 50 microns.

176. The wetted particulate of claim 165, wherein the porous scaffold has an average pore size of 2 to 20 microns.

177. The wetted particulate of claim 160, wherein the scaffold is dehydrothermally cross-linked.

178. The wetted particulate of claim 163, wherein the scaffold is dehydrothermally cross-linked.

179. The wetted particulate of claim 165, wherein the average cross-sectional area, or volume or maximum diameter of wetted particulates are within ±20% of the values for the dry precursors.

180. The wetted particulate of claim 165, wherein the scaffold is dehydrothermally cross-linked.
